(19) United States
(12) Patent Application Publication
(10) Pub. No.: US 2015/0030709 A1
(43) Pub. Date: Jan. 29, 2015

(54) CARDIO-PROTECTIVE AGENTS FROM KIWIFRUITS

(71) Applicant: University of Oslo, Oslo (NO)

(72) Inventor: Asim Kanti Duttaroy, Oslo (NO)

(73) Assignee: University of Oslo, Oslo (NO)

(21) Appl. No.: 14/379,901

(22) PCT Filed: Feb. 20, 2013
(86) PCT No.: PCT/US2013/026826

§ 371 Date: Aug. 20, 2014

Related U.S. Application Data
(60) Provisional application No. 61/601,274, filed on Feb. 21, 2012.

(51) Int. Cl. A61K 36/185; A23L 1/30

(52) U.S. Cl. CPC A61K 36/185; A23L 1/3002
USPC 424/777; 426/648

(57) ABSTRACT

The invention relates to cardio-protective agents. In particular, the present invention relates to de-sugared cardio-protective extracts and fractions thereof prepared from kiwi fruit.

### FIELD OF THE INVENTION

The invention relates to cardio-protective agents. In particular, the present invention relates to de-sugared cardio-protective extracts and fractions thereof prepared from kiwi fruit.

### BACKGROUND OF THE INVENTION

It is known that a high consumption of fruits and vegetables is an important preventive measure by which risk of cardiovascular diseases and certain nutritionally linked cancers including stomach, colon, breast, and prostate cancer can be decreased. One factor involved in the initiation and development of both cardiovascular diseases and cancers is the occurrence of abnormal oxidative stress processes leading to the generation of hydroxy and peroxy free radicals or compounds. In part, the beneficial effect of eating fruits and vegetables is explained by the antioxidants known to account for the inhibition include vitamin C, vitamin E and carotenoids such as alpha and beta carotenoids, lycopene lutein, etc. However, many emerging data also indicates a role for non-antioxidant properties of some compounds in fruits in different diseases.

Considerable effort has been expended in identifying bioactive compounds derived from fruits and vegetables may have a role in the prevention of some diseases. Fruits and vegetables have been thought to be beneficial in cardiovascular disease. The beneficial effects of fruits and vegetables may be explained by antioxidants and bioactive non-antioxidant components contained therein. These compounds may function individually or in concert to protect lipoproteins and vascular cells from oxidation, or by other mechanisms (non-antioxidant pathways) such as reducing plasma lipid levels (LDL cholesterol, triglycerides), and platelet aggregation response (26,27).

Additional preparations from fruits and vegetables that provide cardio-protective and other beneficial properties are needed.

### SUMMARY OF THE INVENTION

The invention relates to cardio-protective agents. In particular, the present invention relates to de-sugared cardio-protective extracts and fractions thereof prepared from kiwi fruit.

In some embodiments, the present invention provides a composition comprising a fruit extract from a fruit of the family Actinidia, said extract characterized as being de-sugared and having a biological activity. In some embodiments, the compositions comprises compounds that have a decreased solubility in an aqueous solvent or alcohol as compared to fructose. In some embodiments, the extract comprises less than about 30% w/w sugars. In some embodiments, the extract comprises less than about 5% w/w sugars. In some embodiments, the extract comprises less than about 1.0% w/w sugars. In some embodiments, the extract comprises less than about 0.5% w/w sugars. In some embodiments, the extract is characterized in being substantially free of sugars. In some embodiments, the composition is stable. In some embodiments, the composition retains biological activity during storage. In some embodiments, the fruit extract is stabilized by a method selected from the group consisting of ultrafiltration, heat treatment, and combinations thereof. In some embodiments, the heat treatment comprises heating to at least 70, 80, 90, 100, 110, 120, or 130 degrees Celsius and up to about 135 degrees Celsius.

In some embodiments, the biological activity is inhibition of platelet aggregation in an in vitro platelet aggregation assay. In some embodiments, the extract has more than 4% inhibitory activity in an in vitro platelet aggregation assay after kept at 4 degrees Celsius for 24 days normalized to day 0. In some embodiments, the composition is further characterized in retaining at least 80% of biological activity of said biologically active molecules when stored for 4 days at 4 degrees Celsius as compared to a fresh extract fraction, wherein said biological activity is inhibition of platelet aggregation in an in vitro platelet aggregation assay. In some embodiments, the composition is further characterized in retaining at least 80% of biological activity of said biologically active molecules when stored for at least 18 days at 4 degrees Celsius as compared to a fresh extract fraction, wherein said biological activity is inhibition of platelet aggregation in an in vitro platelet aggregation assay. In some embodiments, the composition is further characterized in retaining at least 80% of biological activity of said biologically active molecules when stored for at least 24 days at 4 degrees Celsius as compared to a fresh extract fraction, wherein said biological activity is inhibition of platelet aggregation in an in vitro platelet aggregation assay. In some embodiments, the biological activity is inhibition of angiotensin-converting enzyme.

In some embodiments, the extract is delipidated. In some embodiments, the extract is characterized by consisting essentially of biologically active molecules with a molecular weight of less than 3000 daltons. In some embodiments, the extract is characterized by consisting essentially of biologically active molecules with a molecular weight of less than 1000 daltons. In some embodiments, the extract is characterized by consisting essentially of biologically active molecules with a molecular weight of less than 3000 daltons. In some embodiments, the extract is characterized by consisting essentially of biologically active molecules with a molecular weight of less than 1000 daltons.

In some embodiments, the fruit extract exhibits major peaks at approximately 8.38 and 9.25 minutes on a UV spectrum scan of liquid chromatography of said extract on a Zorbax 1.8 μM particle rapid resolution C18 column (4.6 mm×50 mm, 1.8 μm) with a 100% mobile phase (A) water-formic acid (100:0.1, v/v/v) to 100% B acetonitrile-formic acid (100:0.1, v/v/v) during 35 minutes. In some embodiments, the extract exhibits major UV spectrum peaks as observed in FIG. 10. In some embodiments, the fruit extract exhibits major peaks at approximately 30.26, and 30.71 in a mass spectometry 100-1000 Mw in positive mode scan of liquid chromatography of said extract on a Zorbax 1.8 μM particle rapid resolution C18 column (4.6 mm×50 mm, 1.8 μm) with a 100% mobile phase (A) water-formic acid (100:0.1, v/v/v) to 100% B acetonitrile-formic acid (100:0.1, v/v/v) during 35 minutes. In some embodiments, the extract exhibits major total ion current chromatogram peaks as observed in FIG. 8. In some embodiments, the fruit extract exhibits a major peak at approximately 30.79 in a mass spectometry 100-1000 Mw in negative mode scan of liquid chromatography of said extract on a Zorbax 1.8 μM particle rapid resolution C18 column (4.6 mm×50 mm, 1.8 μm) with a 100% mobile phase (A) water-formic acid (100:0.1, v/v/v) to 100% B acetonitrile-formic acid (100:0.1, v/v/v) during 35 minutes. In some embodiments, the extract exhibits major total ion current chromatogram peaks as observed in FIG. 9.

In some embodiments, the present invention provides a syrup or solution comprising a composition as described above. In some embodiments, the present invention provides a powder comprising a composition as described above. In some embodiments, the present invention provides an oral delivery vehicle comprising the composition, syrup, solution or powder as described above. In some embodiments, the present invention provides a functional food or foodstuff comprising the composition, syrup, solution or powder as described above. In some embodiments, the functional food or foodstuff is selected from the group consisting of beverages, baked goods, puddings, dairy products, confections, snack foods, frozen confections or novelties, prepared frozen meals, candy, snack products, soups, spreads, sauces, salad dressings, prepared meat products, cheese, and yogurt. In some embodiments, the present invention provides a nutritional supplement comprising the composition, syrup, solution or powder as described above. In some embodiments, the nutritional supplement is selected from the group consisting of soft gel capsules, hard shell capsules, chewable capsules, health bars, and supplement powders.

In some embodiments, the present invention provides methods of preventing or treating a disease state initiated or characterized by platelet activation and/or aggregation, improving or maintaining heart health, improving or maintaining cardiovascular health, improving or maintaining circulatory health, or improving or maintaining blood flow in a subject comprising administering to said subject a composition, syrup, powder, oral delivery vehicle or nutritional supplement, functional food or foodstuff as described above. In some embodiments, the administering of the composition inhibits platelet aggregation. In some embodiments, the administering of the composition results in anti-thrombotic activity. In some embodiments, the administering of the composition results in blood thinning. In some embodiments, the administering of the composition results in reduced blood pressure.

In some embodiments, the present invention provides for the use of the composition, syrup, powder, oral delivery vehicle or nutritional supplement, functional food or foodstuff according as described above for preventing or treating a disease state initiated or characterized by platelet activation and/or aggregation, improving or maintaining heart health, improving or maintaining cardiovascular health, improving or maintaining circulatory health, or improving or maintaining blood flow in a subject, or improving or maintaining blood pressure in a subject. In some embodiments, the disease state initiated or characterized by platelet activation and/or aggregation is selected from the group consisting of thrombosis, arteriosclerosis and and/or plaque formation.

In some embodiments, the present invention provides processes for producing a stable and biologically active Actinidia extract comprising producing an Actinidia extract, heating the Actinidia extract under conditions such that the extract retains biological activity during storage, and de-sugaring the Actinidia extract before or after said heating. In some embodiments, the de-sugaring is performed by a process selected from the group consisting of solid-phase extraction, fermentation, enzyme treatment and nanofiltration. In some embodiments, the heating comprises heating said fraction to about 70 to about 100 degrees Celsius for greater than about five minutes. In some embodiments, the Actinidia extract is produced by sedimenting an Actinidia juice or homogenate either before or after heating to provide a sediment fraction and a supernatant fraction, and retaining said supernatant fraction to provide said biologically active Actinidia extract. In some embodiments, the sedimentation comprises centrifugation at least 3000 g. In some embodiments, the extract is additionally processed by ultrafiltration either before or after heating. In some embodiments, the ultrafiltration has a cut-off of between 1000-3000 Daltons.

In some embodiments, the present invention provides the desugared, stable extract produced by the processes described above.

In some embodiments, the present invention provides a method of reducing blood pressure or treating hypertension in a subject comprising administering to a subject in need thereof an effective amount of kiwi fruit or kiwi fruit extract, wherein said effective amount comprises greater than about one whole fruit equivalents of kiwi fruit. In some embodiments, the effective amount comprises greater than about 3 whole fruit equivalents of kiwi fruit. In some embodiments, the effective amount comprises from about 2 to about 10 whole fruit equivalents of kiwi fruit. In some embodiments, the effective amount comprises from about 3 to about 5 whole fruit equivalents of kiwi fruit. In some embodiments, the kiwi fruit extract is selected from the group consisting of concentrates, powders, syrups, and de-sugarized extracts prepared from kiwi fruit. In some embodiments, the effective amount causes a reduction in blood pressure in said subject when administered over a time frame selected from the group consisting of 1 week, 2 weeks, 3, weeks, 4 weeks, 5 weeks, 10 weeks, 20 weeks, 30 weeks, 40 weeks, and 50 weeks.

In some embodiments, the present invention provides for use of an effective daily dosage of kiwi fruit or kiwi fruit extract, wherein said effective daily dosage comprises greater than about 1 whole fruit equivalents of kiwi fruit for the treatment of hypertension or reduction of blood pressure in a subject. In some embodiments, the effective daily dosage comprises greater than about 3 whole fruit equivalents of kiwi fruit. In some embodiments, the effective daily dosage comprises from about 2 to about 10 whole fruit equivalents of kiwi fruit. In some embodiments, the effective daily dosage comprises from about 3 to about 5 whole fruit equivalents of kiwi fruit. In some embodiments, the kiwi fruit extract is selected from the group consisting of concentrates, powders, syrups, and de-sugarized extracts prepared from kiwi fruit. In some embodiments, the effective daily dosage causes a reduction in blood pressure in said subject when administered over a time frame selected from the group consisting of 1 week, 2 weeks, 3, weeks, 4 weeks, 5 weeks, 10 weeks, 20 weeks, 30 weeks, 40 weeks, and 50 weeks.

### DESCRIPTION OF THE FIGURES

FIG. 1 shows in schematic from a procedure for partial fractionation of kiwi fruit extracts.

FIG. 2 shows platelet aggregation inhibition induced by ADP activity by the extract.

FIG. 3 shows the inhibition of platelet aggregation induced by arachidonic acid.

FIG. 4 shows the effects of KFE on ACE activity of human serum.

FIG. 5 shows UV scanning of a delipidated, ultrafiltrated purified fraction of kiwifruit extract.

FIG. 6 provides a chromatogram of a UV spectral 200-400 nm scan of the kiwi extract of FIG. 5.

FIG. 7 provides a chromatogram of a MS scan 100-1000 Mw in negative mode of the kiwi extract of FIG. 5.

FIG. 8 provides a chromatogram of a MS scan 100-1000 Mw in positive mode of a desugared, delipidated, ultrafiltrated purified fraction of kiwifruit extract.

FIG. 9 provides a chromatogram of a MS scan 100-1000 Mw in negative mode of a desugared, delipidated, ultrafiltrated purified fraction of the kiwifruit extract of FIG. 8.

FIG. 10 provides a chromatogram of a UV spectral 200-400 nm scan of a desugared, delipidated, ultrafiltrated purified fraction of the kiwifruit extract of FIG. 8.

FIG. 11 shows the strong inhibitory effect of methanolic eluates at different concentrations on ADP-induced platelet aggregation.

FIG. 12A is a dose response curve for ACE inhibitory activity of a kiwifruit extract of the present invention. FIG. 12B is a dose response curve for ACE inhibitory activity of a synthetic agent, captopril.

### DEFINITIONS

As used herein the term ‘fraction’ refers to a partially purified extract or compounds purified from an extract.

As used herein, the term “sugars” refers to water-soluble monosaccharides and disaccharides present in fruits.

As used herein, the term “de-sugared” refers to a composition from which water-soluble monosaccharides and disaccharides have been at least partially removed.

As used herein, the term “whole fruit equivalent” refers to an amount of fruit or extract, such as a juice, powder, de-sugared extract, etc. that contains an equivalent amount of activity (e.g., platelet aggregation inhibiting activity, blood pressure lowering activity, or ACE inhibiting activity) or active ingredients as compared to a whole fruit. In the case of kiwi fruit, a whole fruit equivalent corresponds to a kiwi fruit having an average weight of approximately 65 grams.

The term “purified” or “to purify” means the result of any process that removes some of a contaminant from the component of interest, such as the components responsible for inhibition of platelet aggregation. The percent of a purified component is thereby increased in the sample.

As used herein, the term “physiologically acceptable carrier” refers to any carrier or excipient commonly used with oily pharmaceuticals. Such carriers or excipients include, but are not limited to, oils, starch, sucrose and lactose.

As used herein, the term “oral delivery vehicle” refers to any means of delivering a pharmaceutical orally, including, but not limited to, capsules, pills, tablets and syrups.

As used herein, the term “food product” refers to any food or feed suitable for consumption by humans, non-ruminant animals, or ruminant animals. The “food product” may be a prepared and packaged food (e.g., mayonnaise, salad dressing, bread, or cheese food) or an animal feed (e.g., extruded and pelleted animal feed or coarse mixed feed). “Prepared food product” means any pre-packaged food approved for human consumption.

As used herein, the term “foodstuff” refers to any substance fit for human or animal consumption.

As used herein, the term “functional food” relates to any fresh or processed food claimed to have a health-promoting and/or disease-preventing property beyond the basic nutritional function of supplying nutrients. Functional foods are sometimes called nutraceuticals. The general category includes processed food made from functional food ingredients, or fortified with health-promoting additives, like “vitamin-enriched” products, and also, fresh foods (e.g., vegetables) that have specific claims attached. Fermented foods with live cultures are often also considered to be functional foods with probiotic benefits.

As used herein, the term “nutritional supplement” refers to a food product formulated as a dietary or nutritional supplement to be used as part of a diet.

### DETAILED DESCRIPTION OF THE INVENTION

The invention relates to cardio-protective agents. In particular, the present invention relates to de-sugared cardio-protective extracts and fractions thereof prepared from kiwi fruit.

Kiwifruit is the most well-known crop in the genus Actinidia (3). Although Actinidia fruit sales in the international market are dominated by a single kiwifruit cultivar Actinidia deliciosa “Hayward,” there are a considerable number of cultivars and selections in the genus that have widely diverse shape, size, and hairiness. They also offer a wide variation in sensory attributes such as flesh color, flavor, and taste, and in nutritional attributes such as the vitamin C level, polyphenols, and carotenoid content (29, 30). Few types of processed kiwifruit food products are commercially available to consumers. Kiwifruits are mainly eaten as whole fruits. The few examples where kiwifruit has been processed into products include frozen desserts and blended juices and more recently a few natural kiwifruit drinks such as Kiwi Crush™ (Vital Food Processors Ltd, Manukau City, Auckland, New Zealand). Kiwifruit extracts containing the fruit's nutritional components and desirable bioactives, including polyphenols, ascorbic acid and water-soluble polysaccharides (pectic polysaccharides), which may be advantageous for functional food applications, increasing the range of kiwifruit products available to consumers (28). With growing health awareness, there are increased consumer demands for acceptable nutritional foods with multiple consumer benefits including defined health benefits, increased convenience and reduced additives.

Platelets are involved in the development of atherosclerosis, and thrombotic events, and therefore reduction of platelet activity by medications reduces the incidence and severity of disease (26). Experiments conducted during the course of development of embodiments of the present invention evaluated whether consuming kiwi fruit modulated platelet activity and plasma lipids in healthy human volunteers in a randomized crossover study. It was reported that consuming two or three kiwi fruits per day for 28 days reduced platelet aggregation response to collagen and ADP by 18% compared with the controls (7). In addition, consumption of kiwi fruits lowered blood triglycerides levels by 15% compared with control, whereas no such effects were observed in the case of plasma cholesterol levels. All these data indicate that consuming kiwi fruit is beneficial in cardiovascular disease. Incubation of kiwi fruit extract (KFE; expressed as weight of pulp used to prepare KFE) inhibited platelet aggregation but was not an optimal preparation as it requires a good amount of flesh plus the activity is lost in storage even at 4 degrees C. due to unwanted reactions in the juice. In addition, it is thought that tannins and oil in the seeds (and to a lesser extent the hair) can react with the highly acidic pulp to give rise unwanted smell and color. Many kiwifruit species have a fine hair which is difficult to remove from a juice. Soft-pulping methods are preferred as it is considered desirable to avoid both excessive cell disintegration and fragmenting components of the fruit such as the seed. Seeds may contain toxic substances (e.g. apricot kernel) or contribute to off or undesirable flavors in a juice.

Many fruits are acidic and those with a pH of 6 or less are generally most likely to be affected. Widely used and relatively inexpensive sucrose is alkaline and appears to induce or take part in further undesirable reactions when added to an acidic pulp. Research leading to embodiments of the present invention indicated that substances entering a juice from seed fragmentation or excessive cell damage contribute to factors adversely affected the production of successful kiwifruit juice, such as problems of browning and catch factor. The kiwifruit is more acidic than most and has a pH of approximately 3. This may also avoid any possible side reactions contributing to catch, discoloration etc. Glucose and fructose are commonly found in many fruits. Typically this is by masking some of the undesirable properties of fruit such as bitterness or excess acidity and is due partly to the average human's affinity for sweetness. In some embodiments, juice products have not been pasteurized as characteristic of most other juices and processes. Hence any preservative effects which are contributed by the sweetening agent will help prolong the shelf life of the product. It is possible that the alteration in pH resulting from the combination may cause unwanted side reactions. Furthermore, it is noted that under storage, the chemistry of most juices will vary. For kiwifruit, the acid content of the juice will drop. Accordingly, in some embodiments the addition of a suitable buffering or pH adjusting agent help to preserve the pH of the product over a longer period. This also defers any undesirable long term reactions resulting in browning or discoloration of the juice.

In some embodiments, the active fractions of fruit, and in particular kiwifruit, are utilized in a variety of formulations and are preferably added to any matrix for human consumption that as are known in the art. In some preferred embodiments, the active fractions are characterized in having high efficacy for a particular use, such as prevention of platelet aggregation or adhesion, as being substantially free from inactive materials, as having an enhanced shelf-life as compared to untreated active fractions. In some embodiments, the fractions are produced by a process where a juice or pulp fraction is centrifuged, filtered, and delipidated to provide highly enriched platelet inhibitors (i.e., more than 10, 20, or 30 fold and up to about 50 fold or 100 fold as compared with raw, unprocessed juice). This process produces an active fraction with an enhanced shelf-life and which is stable to heating. In some preferred embodiments, the active fraction is heat-treated to further enhance stability.

In some embodiments, the extracts are de-sugared either before or after the processing steps described above. Surprisingly, the active components present in the kiwi extracts are less water-soluble than sugar and are retained in alcohol. In some preferred embodiments, the extracts are de-sugared by column chromatography, and in particularly preferred embodiments by solid phase extraction column chromatography. In other embodiments, sugars are removed by an alternative method, for example by fermentation, enzyme treatment or nanofiltration. Sugars that are removed include sucrose and fructose. In some embodiments, the de-sugared extracts are characterized in comprising less than about 50%, 40%, 30%, 20%, 10, or 5% w/w sugars, preferably less than about 1% w/w sugars, more preferably less than about 0.5% w/w sugars, and most preferably less than about 0.1% w/w sugars. In some embodiments, the de-sugared extracts are characterized in being substantially free of sugars. It will be recognized that the de-sugared extracts are suitable for making powders by known techniques and for inclusion in foods, nutritional supplements, dietary supplements and oral delivery vehicles that are suitable for administration to diabetics as well as non-diabetics.

In further embodiments, there is provided a reconstituted product from an active fraction as described above. The present invention has been developed for members of the genus Actinidia. Fruit products, other than a juice, are also within the scope of embodiments of the invention. These fruit generally have a low pH (3.0-3.5), suffer from browning upon exposure of a juice to air and have a chlorophyll content. It is envisaged that while the process of the invention will be amenable to other fruit, the greatest advantage is likely to be realized for fruit suffering problems and characteristics in common with the kiwifruit e.g. a pH of less than 4.5, significant chloroplast levels, or catch (e.g. the fruit of Monstera deliciosa). It should not be inferred that benefit from the invention is limited to these types of fruit.

In sum, the invention has identified several problem areas, especially for kiwifruit, and addresses their needs.

Experiments conducted during the course of developments of embodiments of the present invention demonstrated that the KFE exhibits an ability to inhibit platelet aggregation, and reduce angiotensin converting enzyme (ACE) activity in vitro. The results obtained to date indicate that compositions containing KFE are of use in preventing cardiovascular disease, for example myocardial infarctions, and stroke and in preventing further thrombo-embolic events in patients who have suffered myocardial infarction, stroke or unstable angina. In addition such composition is of use in preventing restenosis following angioplasty and bypass procedures. Moreover KFE is of use in the treatment of coronary disease resulting from thrombo-embolic disorders such as MI in conjunction with thrombolytic therapy. Results obtained to date indicate that compounds responsible for anti-platelet aggregation activity are water soluble compounds having a very different structure to the lipid soluble compounds. There are many known anti-platelet aggregating agents that act different stages platelet production and action. Aspirin (acetylsalicylic acid) is the most widely used and studied. Dipyridamole and ticlopidine have also been used. Aspirin's anti-platelet activity is due to irreversible inhibition of platelet cyclooxygenase, thus preventing the synthesis of thromboxane A₂, a compound that causes platelet aggregation. Ibuprofen is a reversible inhibitor of platelet cyclooxygenase. Some compounds are direct inhibitors of thromboxane A₂ synthetase, for example pirmagrel, or act as antagonists at thromboxane receptors, for example sulotroban.

The present invention is not limited to a particular mechanism. Indeed, an understanding of the mechanism is not necessary to practice the present invention. Nonetheless, the results described herein indicate that the active components in the fruit extract may affect one or more steps of the pathways leading to the production of thromboxane A₂ upstream from that of aspirin and other anti-platelet drugs currently available. It is well known that the adverse effects are common occurrences with therapeutic doses of aspirin; the main effects being gastro-intestinal disturbances such as nausea, dyspepsia, and vomiting. It is anticipated therefore that the isolated platelet aggregation inhibition compounds in fruit extract find use in as a desirable alternative to aspirin and other anti-platelet drugs in the prevention of thromboembolic events and coronary disease.

Accordingly, in some embodiments, the invention provides a fruit extract, active fraction thereof, or one or more active compounds isolatable therefrom, for use in the prophylaxis or treatment of a disease state initiated or characterized by platelet aggregation.

In further embodiments, the invention provides a fruit extract or active fraction thereof or one or more compounds isolatable thereof for use as an anti-thrombotic agent.

In still further embodiments, the invention provides a fruit extract or active fraction thereof or one or more compounds isolatable thereof as here in before defined for the manufacture of a medicament for use in the prophylaxis or treatment of a disease state initiated or characterized by platelet aggregation; or for use as a platelet aggregation inhibitor: or for use an anti-thrombotic agent.

In some embodiments, the invention provides a process for the manufacture of a medication for use (i) in the prophylaxis or treatment of a disease state initiated, mediated or characterized by platelet aggregation, or (ii) as a platelet aggregation inhibitor, or as (iii) an anti-thrombotic agent: which process is characterized by the use, as an essential ingredient of the medicament, of a fruit, or an extract or active fraction thereof or one or more active components isolatable thereof as hereinbefore defined.

In some embodiments, the invention provides a pharmaceutical composition comprising an active component derived from a fruit or an extract or active fraction or one or more active compounds isolatable thereof as hereinbefore defined and pharmaceutically acceptable carrier.

In some embodiments, the invention provides a fruit extract, active fraction thereof, or one or more active compounds isolatable therefrom, for use in supporting cardiovascular health.

In some embodiments, the invention provides a fruit extract, active fraction thereof, or one or more active compounds isolatable therefrom, for use in supporting heart health.

In other embodiments, the invention provides a fruit extract or active fraction thereof or one or more compounds isolatable thereof for use as a platelet aggregation inhibitor.

In other embodiments, the invention provides a fruit extract or active fraction thereof or one or more compounds isolatable thereof for use in promoting or maintaining heart health and/or circulatory health.

In other embodiments, the invention provides a fruit extract or active fraction thereof or one or more compounds isolatable thereof for use in improving, maintaining and or promoting blood flow, and in particular the smooth flow of blood.

It is preferred that the fruit extract used in accordance with the invention are those which are non toxic to humans and typically the fruits which are usually considered to be edible fruits. Thus the fruits may or may not contain seeds or stones but have an edible essentially non-oily flesh.

Kiwifruit is the most well-known crop in the genus Actinidia. The extracts of embodiments of the invention can be prepared by homogenising the flesh of a peeled kiwifruit and then removing solids therefrom, for example by means of centrifugation. Thus the extract is a typically an aqueous extract, which can consist or comprise the juice of the fruit, optionally with the addition of extra water added during the homogenising step. Such aqueous extracts can be concentrated, enriched or condensed by, for example, standard techniques, e.g. evaporation under reduced pressure. Examples of concentrates are those which are at least 2-fold concentrated, more usually, at least 4-fold, for example at least 8-fold, or at least 40-fold or at least 100-fold or at least 200 fold or at least 1000 fold.

The extract can be fractionated to isolate one or more active fractions therein by, for example, molecular weight filtration, or chromatography on suitable support such as sepharose gel (for size exclusion chromatography) or removal of lipids (by Lipidex-1000) or by solvent treatments, or ion exchange column using HPLC on a suitably treated silica or alumina, for example ODS coated silica, or solvent extraction.

Experiments carried out on kiwi fruit extract have revealed that the active components of the extract passes through an ultrafiltration having molecular weight cut-off of 1000 is colorless, water soluble and does not lose activity when boiled. In some embodiments, the present invention provides a process for producing a stable and biologically active Actinidia extract comprising fractionating juice from an Actinidia fruit to produce an extract fraction and heating the extract fraction to about 70 to about 120 degrees Celsius, preferably 80 to 100 degrees Celsius, and most preferably to about 95 to 100 degrees Celsius. In some embodiments, the duration of the heating is from about 5 to about 30 minutes, preferably about 10 to about 25 minutes, and most preferably about 20 minutes, or more for more than about 5, 10, or 15 minutes. In some embodiments, the present invention provides a process for producing a stable and biologically active Actinidia extract comprising fractionating juice from an Actinidia fruit to produce an extract fraction and subjecting the fraction to ultrafiltration with a molecular weight cutoff of less than 10 kDa, preferably less than 5 kDa, and more preferably less than about 3 kDa, 2 kDa or 1 kDa. In some embodiments, the stabilized active fraction comprises biologically active molecules and is characterized in retaining at least 80% of biological activity of said biologically active molecules when stored for at least 4 days, 18 days or 24 days up to about 30 or 40 days at 4 degrees Celsius as compared to a fresh extract fraction. In some embodiments, the biological activity is inhibition of platelet aggregation in an in vitro platelet aggregation assay or inhibition of angiotensin converting enzyme activity.

In some preferred embodiments, the stable and biologically active Actinidia extract produced by this method exhibits major peaks at approximately 1.30 and 1.81 minutes on a UV spectrum chromatogram and major peaks at approximately 1.61, 30.18, and 30.87 on a total ion current chromatogram and wherein said extract inhibits platelet aggregation in an in vitro platelet aggregation assay.

Accordingly, embodiments of the invention also provides for use an antithrombotic agent, or for use as a platelet aggregation inhibitor, or for use in the prophylaxis or treatment of a disease state initiated or characterized by platelet aggregation, an active fraction of a fruit extract (e.g., kiwifruit extract) the active fraction containing a substantially heat stable colorless or slightly straw colored water soluble compounds with a molecular weight less than 3000, 2000, or 1000 kDa. In some embodiments, the active fraction is characterized as having a biologically activity. In some embodiments, the biological activity is an inhibition or decrease of angiotensin converting enzyme (ACE) activity by at least 5%, 10% or preferably 15% as compared to a control or placebo substance when the active fraction is incubated with normal serum for 10 minutes. In some embodiments, the biological activity is reduction of blood pressure by at least 1, 5, 10, 15 or up to 20 mm Hg in the systolic or diastolic measurement or a combination thereof. In some embodiments, the biological activity is inhibition of platelet aggregation in an in vitro platelet aggregation assay. In some embodiments, the platelet aggregation inhibition is expressed as a percent inhibition of platelet aggregation by a known effector of platelet aggregation, for example collagen, ADP, or arachidonic acid. In some embodiments, the active fraction of the present invention inhibits platelet aggregation by one of these known effectors by at least 10%, 20%, 30% 40% or 50% up to about 50% or 60% as compared to a control or placebo substance.

The active fraction has been found to be primarily associated with, or extractable from, the juice, the flesh surroundings the pips and the pips of the kiwifruit. Thus, the use of compositions prepared from an active fraction consisting essentially of or comprising a homogenate or an extract thereof derived from the flesh of a peeled kiwifruit or consisting essentially of or comprising the juice and/or the flesh surrounding the pips, and or the pips, represents a preferred embodiment of the invention.

Accordingly, embodiments of the present invention provide an active fraction of a kiwifruit extract with one or more of the following characteristics:
a) The size of molecules in the active fraction is less than 3000 kDa, and preferably less than 2000 kDa or 1000 kDa;
b) The active fraction is substantially heat stable;
c) The active fraction is substantially colorless;
d) The active fraction substantially comprises water soluble compounds;
e) The active fraction inhibits platelet aggregation; and
f) The active fraction inhibits angiotensin converting enzyme

The active fractions of the present invention may be provided in a variety of forms and in a variety of formulations. In some embodiments, the fractions are provided in as a liquid, a syrup, a powder, a paste, an emulsion, a pelleted composition, a granulated composition, an encapsulated composition, a suspension, a concentrate, a solution, and a lozenge. The powders may preferably be a lyophilized, freeze dried or spray dried powder prepared from the stabilized kiwi extract with or without a organoleptically and/or pharmaceutically acceptable excipient. The syrups may preferably be a viscous, concentrated aqueous solution prepared from the stabilized kiwi extract and may include suitable excipients and/or sweeteners. The syrups may be utilized for direct oral administration or as a concentrate for reconstitution with water prior to administration.

The fractions may be provided by any of a number of routes, including, but not limited to, oral, intravenous, intramuscular, intra-arterial, intramedullary, intrathecal, intraventricular, transdermal, buccal, subcutaneous, intraperitoneal, intranasal, enteral, topical, sublingual or rectal means. For details on techniques for formulation for and administration and administration may be found in the latest edition of Remington's Pharmaceutical Sciences (Maack Publishing Co., Easton, Pa.).

In some embodiments, the present invention provides an oral delivery vehicle comprising a fraction of the present invention. The fractions may preferably be formulated with pharmaceutically acceptable carriers such as starch, sucrose or lactose in tablets, pills, dragees, capsules, gel capsules, solutions, liquids, slurries, suspensions and emulsions. The tablets or capsules of the present invention may be coated with an enteric coating which dissolves at a pH of about 6.0 to 7.0. A suitable enteric coating which dissolves in the small intestine but not in the stomach is cellulose acetate phthalate. In some embodiments, the oral delivery vehicle comprises an amount of the first and second components effective to cause an effect in subject selected from the group consisting of increasing efficiency of muscle work, decreasing energy cost of work, increasing time of work to exhaustion, increasing endurance during physical exercise, increasing well-being, ameliorating muscle soreness after strenuous exercises, improving metabolic conditions in subjects with obesity and/or metabolic syndrome, and combinations thereof. Examples of improving metabolic conditions in subjects with obesity and/or metabolic syndrome include, but are not limited to, increasing glucose uptake, lowering oxidative stress, and combinations thereof.

In some embodiments, the oral delivery vehicle comprises an effective amount of the fractions. In some embodiments, the effective amount comprises an amount of extract containing the biologically active ingredients found in from about 1 to 10, 1 to 5, 1 to 3, 2 to 4, 2 to 3, about 3 kiwifruits weighing approximately 65 g each. In other embodiments, the effective amount corresponds to about 1 to about 5000 mg of the lyophilized or spray dried, stabilized fraction, preferably from about 1 to about 3000 mg of the lyophilized or spray dried, stabilized fraction and most preferably about 1 mg to about 1000 mg of the lyophilized or spray dried, stabilized fraction. In other embodiments, the effective amount corresponds to about 500 to about 20000 mg of the concentrated (e.g., as syrup), stabilized fraction, preferably from about 500 to about 10000 mg of the concentrated (e.g., as syrup), stabilized fraction and most preferably about 500 mg to about 2500 mg of the concentrated (e.g., as syrup), stabilized fraction. In other embodiments, the effective amount comprises from about 1 to about 5000 mg of the de-sugarized fraction, preferably from about 1 to about 3000 mg of the de-sugarized fraction and most preferably about 1 mg to about 1000 mg of the de-sugarized fraction, or from about 50 mg to 1000 mg, 50 mg to 750 mg, 50 mg to 500 mg, 50 mg to 250 mg 10 mg to 100 mg or 10 mg to 200 mg of the de-sugarized fraction. In some embodiments, the daily dose of kiwi fruit extract contains the active ingredients in the equivalent (i.e., the whole fruit equivalent) of from about 1 to 10, 1 to 5, 1 to 3, 2 to 4, 2 to 3, about 3 kiwifruits weighing approximately 120 g each with peel or 100 g each without peel.

In some embodiments, the present invention provides dietary supplements comprising the fractions of the present invention. The ingredients of the dietary supplement of this invention are preferably contained in acceptable excipients and/or carriers for oral consumption. The actual form of the carrier, and thus, the dietary supplement itself, is not critical. The carrier may be a liquid, gel, gelcap, capsule, powder, solid tablet (coated or non-coated), tea, or the like. The dietary supplement is preferably in the form of a tablet or capsule and most preferably in the form of a soft gelatin capsule. In other embodiments, the supplement is provided as a powder or liquid suitable for adding by the consumer to a food or beverage. For example, in some embodiments, the dietary supplement can be administered to an individual in the form of a powder, for instance to be used by mixing into a beverage, or by stirring into a semi-solid food such as a pudding, topping, sauce, puree, cooked cereal, or salad dressing, for instance, or by otherwise adding to a food. In preferred embodiments, the dietary supplements comprise an effective amount of the components as described above.

The dietary supplement may comprise one or more inert ingredients, especially if it is desirable to limit the number of calories added to the diet by the dietary supplement. For example, the dietary supplement of the present invention may also contain optional ingredients including, for example, herbs, vitamins, minerals, enhancers, colorants, sweeteners, flavorants, inert ingredients, and the like. For example, the dietary supplement of the present invention may contain one or more of the following: asorbates (ascorbic acid, mineral ascorbate salts, rose hips, acerola, and the like), dehydroepiandosterone (DHEA), green tea (polyphenols), inositol, kelp, dulse, bioflavinoids, maltodextrin, nettles, niacin, niacinamide, rosemary, selenium, silica (silicon dioxide, silica gel, horsetail, shavegrass, and the like), spirulina, zinc, docosahexaenoic acid and/or eicosapentaenoic acid (provided in any form such as free fatty acids, trigylcerides or phospholipids) and the like. Such optional ingredients may be either naturally occurring or concentrated forms.

In some embodiments, the dietary supplements further comprise vitamins and minerals including, but not limited to, calcium phosphate or acetate, tribasic; potassium phosphate, dibasic; magnesium sulfate or oxide; salt (sodium chloride); potassium chloride or acetate; ascorbic acid; ferric orthophosphate; niacinamide; zinc sulfate or oxide; calcium pantothenate; copper gluconate; riboflavin; beta-carotene; pyridoxine hydrochloride; thiamin mononitrate; folic acid; biotin; chromium chloride or picolonate; potassium iodide; sodium selenate; sodium molybdate; phylloquinone; vitamin D₃; cyanocobalamin; sodium selenite; copper sulfate; vitamin A; vitamin C; inositol; potassium iodide. Suitable dosages for vitamins and minerals may be obtained, for example, by consulting the U.S. RDA guidelines.

In preferred embodiments, the dietary supplements comprise an effective amount of the fractions as described above. The dietary supplements of the present invention may be taken one or more times daily. Preferably, the dietary supplement is administered orally one to two times daily. Frequency of administration will, of course, depend on the dose per unit (capsule or tablet) and the desired level of ingestion. Dose levels/unit can be adjusted to provide the recommended levels of ingredients per day (e.g., an effective amount as described above) in a reasonable number of units (e.g., two capsules or tablets taken twice a day). In preferred embodiments, the doses add up each day to the daily intake of each ingredient. In preferred embodiments, the dietary supplements are taken with meals or before meals. In other embodiments, the dietary supplements are not taken with meals.

In other embodiments, the present invention provides nutritional supplements (e.g., energy bars or meal replacement bars or beverages) comprising of the fractions of the present invention. In preferred embodiments, the nutritional supplements comprise an effective amount of the components as described above. The nutritional supplement may serve as meal or snack replacement and generally provide nutrient calories. Preferably, the nutritional supplements provide carbohydrates, proteins, and fats in balanced amounts. The nutritional supplement can further comprise carbohydrate, simple, medium chain length, or polysaccharides, or a combination thereof. A simple sugar can be chosen for desirable organoleptic properties. Uncooked cornstarch is one example of a complex carbohydrate. If it is desired that it should maintain its high molecular weight structure, it should be included only in food formulations or portions thereof which are not cooked or heat processed since the heat will break down the complex carbohydrate into simple carbohydrates, wherein simple carbohydrates are mono- or disaccharides. The nutritional supplement contains, in one embodiment, combinations of sources of carbohydrate of three levels of chain length (simple, medium and complex; e.g., sucrose, maltodextrins, and uncooked cornstarch).

Sources of protein to be incorporated into the nutritional supplement of the invention can be any suitable protein utilized in nutritional formulations and can include whey protein, whey protein concentrate, whey powder, egg, soy flour, soy milk soy protein, soy protein isolate, caseinate (e.g., sodium caseinate, sodium calcium caseinate, calcium caseinate, potassium caseinate), animal and vegetable protein and mixtures thereof. When choosing a protein source, the biological value of the protein should be considered first, with the highest biological values being found in caseinate, whey, lactalbumin, egg albumin and whole egg proteins. In a preferred embodiment, the protein is a combination of whey protein concentrate and calcium caseinate. These proteins have high biological value; that is, they have a high proportion of the essential amino acids. See Modern Nutrition in Health and Disease, eighth edition, Lea & Febiger, publishers, 1986, especially Volume 1, pages 30-32.

The nutritional supplement can also contain other ingredients, such as one or a combination of other vitamins, minerals, antioxidants, fiber and other dietary supplements (e.g., protein, amino acids, choline, lecithin, other fatty acids). Selection of one or several of these ingredients is a matter of formulation, design, consumer preference and end-user. The amounts of these ingredients added to the dietary supplements of this invention are readily known to the skilled artisan. Guidance to such amounts can be provided by the U.S. RDA doses for children and adults. Further vitamins and minerals that can be added include, but are not limited to, calcium phosphate or acetate, tribasic; potassium phosphate, dibasic; magnesium sulfate or oxide; salt (sodium chloride); potassium chloride or acetate; ascorbic acid; ferric orthophosphate; niacinamide; zinc sulfate or oxide; calcium pantothenate; copper gluconate; riboflavin; beta-carotene; pyridoxine hydrochloride; thiamin mononitrate; folic acid; biotin; chromium chloride or picolonate; potassium iodide; sodium selenate; sodium molybdate; phylloquinone; vitamin D₃; cyanocobalamin; sodium selenite; copper sulfate; vitamin A; vitamin C; inositol; potassium iodide.

Flavors, coloring agents, spices, nuts and the like can be incorporated into the product. Flavorings can be in the form of flavored extracts, volatile oils, chocolate flavorings, peanut butter flavoring, cookie crumbs, crisp rice, vanilla or any commercially available flavoring. Examples of useful flavoring include, but are not limited to, pure anise extract, imitation banana extract, imitation cherry extract, chocolate extract, pure lemon extract, pure orange extract, pure peppermint extract, imitation pineapple extract, imitation rum extract, imitation strawberry extract, or pure vanilla extract; or volatile oils, such as balm oil, bay oil, bergamot oil, cedarwood oil, walnut oil, cherry oil, cinnamon oil, clove oil, or peppermint oil; peanut butter, chocolate flavoring, vanilla cookie crumb, butterscotch or toffee. In one embodiment, the dietary supplement contains cocoa or chocolate.

Emulsifiers may be added for stability of the final product. Examples of suitable emulsifiers include, but are not limited to, lecithin (e.g., from egg or soy), and/or mono- and di-glycerides. Other emulsifiers are readily apparent to the skilled artisan and selection of suitable emulsifier(s) will depend, in part, upon the formulation and final product.

Preservatives may also be added to the nutritional supplement to extend product shelf life. Preferably, preservatives such as potassium sorbate, sodium sorbate, potassium benzoate, sodium benzoate or calcium disodium EDTA are used.

In addition to the carbohydrates described above, the nutritional supplement can contain natural or artificial (preferably low calorie) sweeteners, e.g., saccharides, cyclamates, aspartamine, aspartame, acesulfame K, and/or sorbitol. Such artificial sweeteners can be desirable if the nutritional supplement is intended to be consumed by an overweight or obese individual, or an individual with type II diabetes who is prone to hyperglycemia.

The nutritional supplement can be provided in a variety of forms, and by a variety of production methods. In a preferred embodiment, to manufacture a health bar, the liquid ingredients are cooked; the dry ingredients are added with the liquid ingredients in a mixer and mixed until the dough phase is reached; the dough is put into an extruder, and extruded; the extruded dough is cut into appropriate lengths; and the product is cooled. The bars may contain other nutrients and fillers to enhance taste, in addition to the ingredients specifically listed herein.

In still further embodiments, the present invention provides food products, prepared food products, or foodstuffs comprising the extracts or fractions described above (i.e., functional foods). In preferred embodiments, the foods comprise an effective amount of the fractions as described above. For example, in some embodiments, beverages and solid or semi-solid foods comprising the extracts, fractions or derivatives thereof are provided. These forms can include, but are not limited to, beverages (e.g., soft drinks, milk and other dairy drinks, and diet drinks), baked goods, puddings, dairy products, confections, snack foods, or frozen confections or novelties (e.g., ice cream, milk shakes), prepared frozen meals, candy, snack products (e.g., chips), soups, spreads, sauces, salad dressings, prepared meat products, cheese, and yogurt.

### EXPERIMENTAL

The following examples are provided in order to demonstrate and further illustrate certain preferred embodiments and aspects of the present invention and are not to be construed as limiting the scope thereof.

### Example 1

### Preparation of Kiwifruit Extract

Extract consisting of 100% kiwifruit juice was prepared. To prepare 100% fruit juice, the fruits were peeled and the flesh was homogenized. The resulting homogenate was spun at 9000×g for 15 min at 4° C. on a centrifuge after which the supernatant was removed and the pH of the juice was adjusted to 7.4 with 1M sodium hydroxide solution. The anti-platelet activity of the kiwi fruit extract (KFE) was determined initially.

Partial Fractionation of Kiwi Fruit Extract.

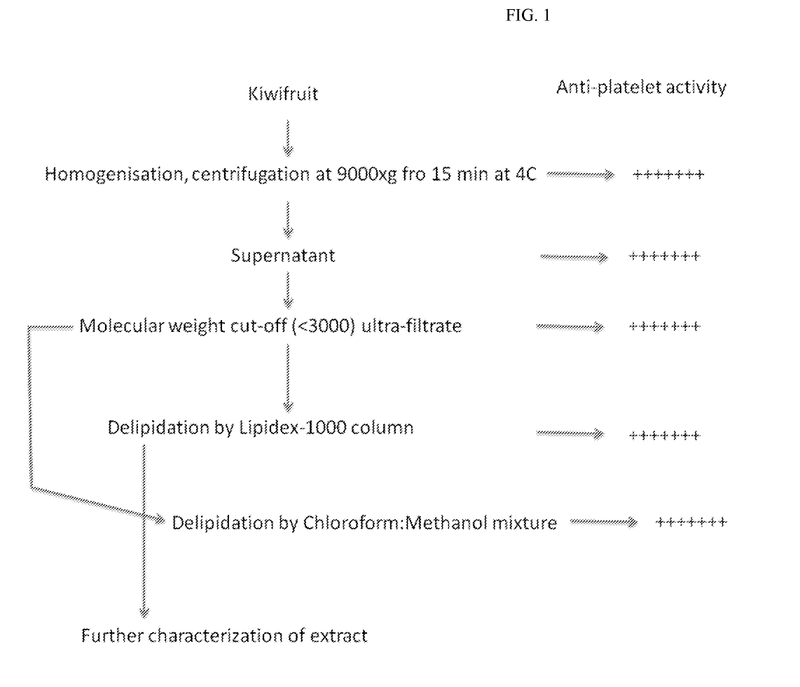

Kiwifruit extracts were then fractionated according to the general scheme set out in FIG. 1. The platelet aggregation inhibiting activity of the preparations was measured at various stages. Thus, fresh kiwifruit juice, prepared from 100% fruit, was centrifuged at 9000×g for 10 min. Following centrifugation, the supernatant was freeze dried and a portion of the dried material was dissolved in phosphate buffer and pH was adjusted to 7.4. This was then subjected to ultrafiltration by passing through an filter with molecular weight cut-off 1000 daltons. The ultrafiltrate was collected, and freeze dried and reconstituted in water, and pH was adjusted to 7.4. The platelet aggregation was measured using the extract at different stages of fractionation. In a separate study, the extract was boiled for 10 min. and centrifuged, and the anti-platelet activity of the boiled sample was determined.

In order to examine whether lipid compounds in the fractionated extracts were responsible for anti-platelet activity, the lipids of the extract were removed by passing the solution through the specially prepared Lipidex-1000 column (column volume 18 ml). Lipidex-1000 adsorbs lipid substances of the extract only. The column was then eluted with 5 column volumes of 15 mM phosphate buffer, and the eluted solution was collected and dried. Lipid compounds bound to column resin were later eluted with methanolic solution and dried for anti-platelet activity measurement. Further to the above Lipidex-1000 experiment, the lipids were also removed with another method by using chloroform methanol according to the Bligh and Dyer. Thus, 2 ml of the ultrafiltrate were mixed with 2.5 ml of methanol followed by 1.25 ml chloroform to give a sing phase, and a chloroform:methanol:water ratio of 1:2:0.8. No precipitate was formed. Chloroform (1.25 mL) and water (1.25 mL) were then added and after gentle mixing, the mixture was allowed to settle into two layers. The upper layer (methanol/water) was removed and the methanol blown off under nitrogen at 55° C. The volume was then made up to 2 mL after adjustment to pH 7.4. The anti-platelet aggregation activity of this aqueous phase was compared with respective volume of phosphate buffer as a control.

The chloroform phase was then evaporated under nitrogen, and resuspended in ethanol (50 μL). A sample (10 μL) of the ethanol phase then tested for anti-platelet aggregation activity versus a 10 μL ethanol control.

Platelet Aggregation Study.

The effect of the fruit extracts on the aggregatory properties of human platelets was investigated in healthy volunteers. Venous blood was collected from volunteers who had not taken any medications for at least 14 days before donation. Blood (20 ml) was collected using a 19 G butterfly needle and coagulation was prevented by mixing the blood samples with acid citrate, (135 mM) in the ration of 9 parts by volume of blood up to 1 part by volume of acid citrate. Platelet rich plasma (PRP) was prepared from the samples by centrifuging the blood at 180×g from 15 min. Kiwi fruit juice (10-30 μl), the pH was adjusted to 7.4 with 1M sodium hydroxide was mixed with the PRP to make volume up to 500 μl, and incubated at 37° C. from 15 min. after which the effect of the fruit extract on ADP induced platelet aggregation was monitored with the addition of ADP to a final concentration 5 μM. Controls were run in parallel using 10-30 μL phosphate buffer, pH 7.4 instead of the fruit extract. Platelet aggregation in PRP was monitored using a Chrono-Log aggregation (Chrono-Log, USA) at a constant stirring speed of 1000 rpm at 37 C.

To determine the effect of KFE on platelet aggregation in vitro, PRP (450 μl) was incubated with different concentrations of KFE (in volume 50 μl for 15 min at 37° C. prior to the addition of an aggregating agent. The IC₅₀ for different fractions of KFE was determined by incubating these platelets with different concentrations of KFE for 15 min. Controls were run in parallel replacing fruit extract with 50 μl of phosphate. Inhibition of platelet aggregation is expressed as the decrease in the area under the curve compared with the control.

Inhibition of Angiotensin Converting Enzyme (ACE) by KFE.

Angiotensin I-converting enzyme (ACE, EC 3.4.15.1), an exopeptidase, is a circulating enzyme that participates in the body's renin-angiotensin system, which mediates extracellular volume (e.g., that of the blood plasma, lymph and interstitial fluid), and arterial vasoconstriction. It is secreted by pulmonary and renal endothelial cells and catalyzes the conversion of decapeptide angiotensin I to octapeptide angiotensin II. ACE inhibitors block the conversion of angiotensin I to angiotensin II. They therefore lower arteriolar resistance and increase venous capacity; increase cardiac output and cardiac index, stroke work and volume, lower renovascular resistance, and lead to increased natriuresis (excretion of sodium in the urine). With ACE inhibitor use, the effects of angiotensin II are prevented, leading to decreased blood pressure. The effect of KFE on the serum ACE activity measured using Angiotensin Converting Enzyme Assay kit by BÜHLMANN LABORATORIES AG, Germany.

### Results:

Fractionation of Kiwi Fruit Extracts and their Effects on ADP-Induced Platelet Aggregation by ADP.

FIG. 1 shows the preparation of kiwifruit extract using different fractionation procedures. The inhibitor(s) of platelet aggregation in kiwi extracts were present in the water soluble fraction and their size is smaller than 1000 daltons. Boiling of this fraction did not destroy the activity. Delipidation of the sample by Lipidex-1000 demonstrated that the active fraction is present in aqueous extract.

Platelet Aggregation Studies.

Table 1 shows the dose response of kiwifruit extract on inhibition of platelet aggregation by different agents. It demonstrated a dose response effect with ADP-induced aggregation: increasing the kiwifruit extract led to greater reduction in platelet aggregation. The fraction isolated from kiwifruit was equally effective against all three platelet aggregating agents, collagen, ADP, and arachidonic acid.

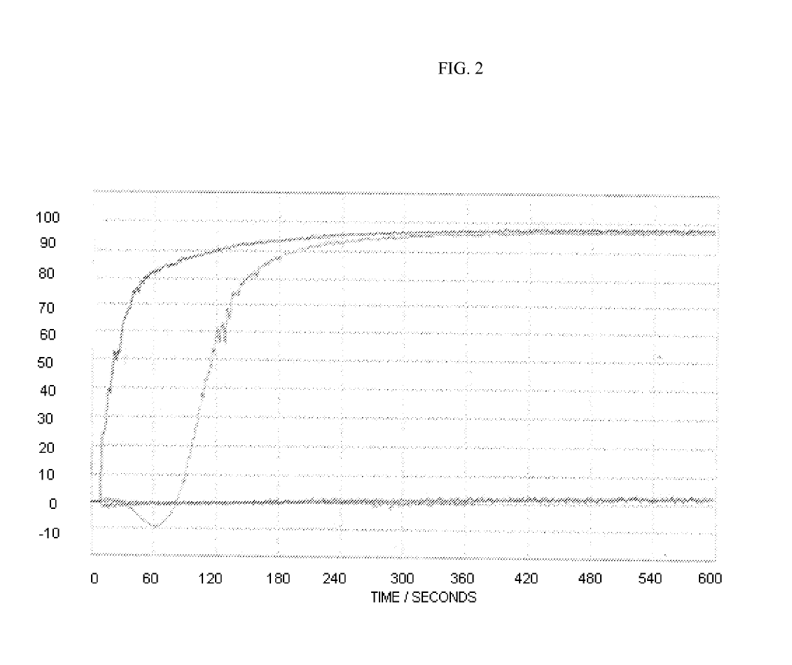

FIG. 2 shows the effect of different volumes of kiwi fruit extract on platelet aggregation by ADP in vitro. PRP (450 ml) was incubated with different volumes (0, 10, 20 and 30 μl) of KFE for 15 min at 37 C prior to the addition of agonists, arachidonic acid (500 μg/m), ADP (3 mM, and collagen (1 μg/ml). KFE inhibited ADP-induced aggregation in a dose dependent manner (Table-1). ADP induced aggregation was inhibited by 45% with 10 μl KFE, 65% with 20 μl KFE, and 95% with 30 μl KFE, compared with controls. Similarly, KFE inhibited collagen induced platelet aggregation; the level of inhibition was lower with 10 and 20 μl incubations. Inhibition of arachidonic acid-induced platelet aggregation exhibited 38% inhibition at the highest KFE level tested and very little inhibition at lower concentrations of KFE.

TABLE 1

| Kiwifruit | Inhibition of platelet aggregation by three different agonists | | |
|---|---|---|---|
| extract | (mean %) | | |
| Volume (μl) | Arachidonic acid | Collagen | ADP |
| 10 | 12 | 18 | 45 |
| 20 | 25 | 45 | 65 |
| 30 | 38 | 90 | 95 |

Boiling of kiwifruit extract at 100 C for 10 min did not affect anti-platelet aggregation of the extract.

Determination of the Effect of KFE on Platelet Aggregation Induced by Different Agonists.

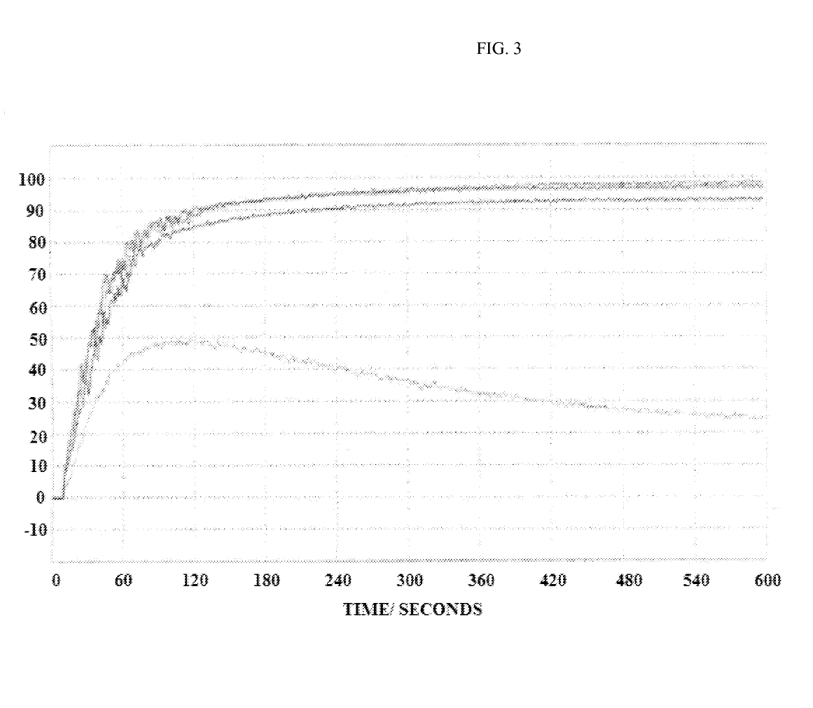

FIGS. 2 and 3 show the inhibition of platelet aggregation induced by arachidonic acid, collagen and ADP, respectively. The experimental conditions are described in Table-1.

Determination of the Effect of Fractionated Kiwifruit Extract on Platelet Aggregation Induced by ADP.

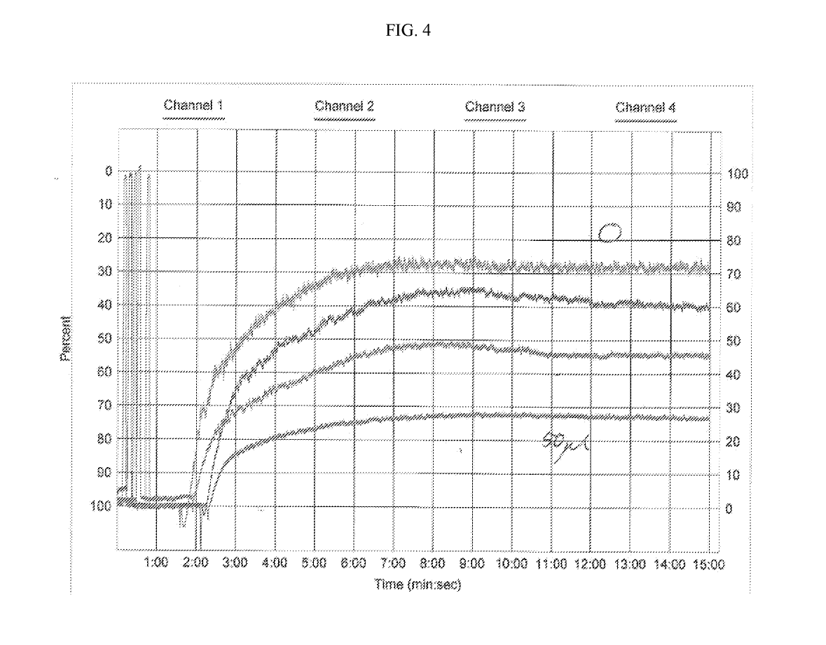

FIG. 4 shows the effect of fractionated kiwifruit extract on platelet aggregation induced by ADP. The experimental conditions are described in Table 1. KFE extract was purified as described in FIG. 1.

Effects of KFE on ACE Activity of Human Serum.

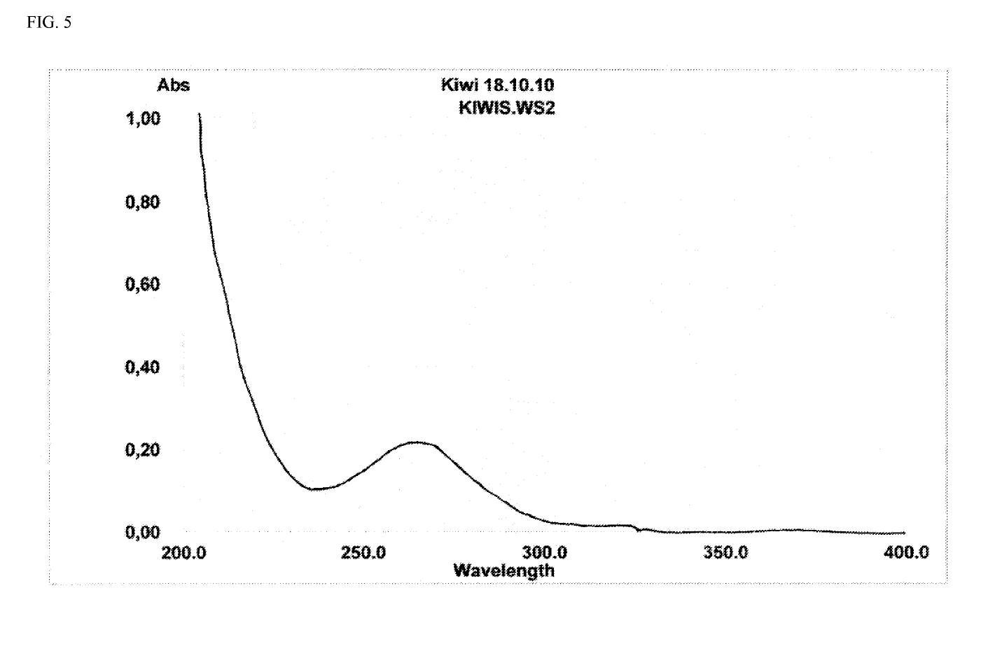

Incubation of serum with 20 μl of KFE for 15 min inhibited more than 15% activity compared with control. FIG. 5 shows UV scanning of the delipidated, ultrafiltrated purified active fractions of the Kiwifruit extract.

### Example 2

Kiwifruit juice was prepared after homogenization of the peeled fruits, subsequently centrifuged at 9000×g for 15 min and kept at 4 C for antiplatelet assay. The other fraction of juice was boiled at 90 C for 20 min and centrifuged again, and pH was adjusted to 7.4 and kept at 4 C up to 24 days.

Inhibition activity of the kiwifruit juice and extract was measured at different days as indicated in the table and their anti-platelet activity was measured by incubating the PRP with the juice (after adjusting pH to 7.4) or the extract for 15 minutes, and the inhibition was compared with control (in the absence of juice or extract) using 3 μM ADP as an aggregating agent.

TABLE 2

| | Kiwifruit Juice | Kiwifruit extract |
|---|---|---|
| Day | Inhibitory Activity | Activity |
| Day 0 | 100 | 100 |
| Day 4 | 76 | 100 |
| Day 18 | 29 | 100 |
| Day 24 | 4 | 100 |

Conclusion: The juice lost activity by 24% within a week and 70% after 18 days, whereas the boiled extract retained its 100% activity. The freeze-dried extract retained its activity as well.

### Example 3

This example describes the UV and MS spectra of the highly purified heat stable and water soluble kiwi fruit extract that contains anti-platelet activity.

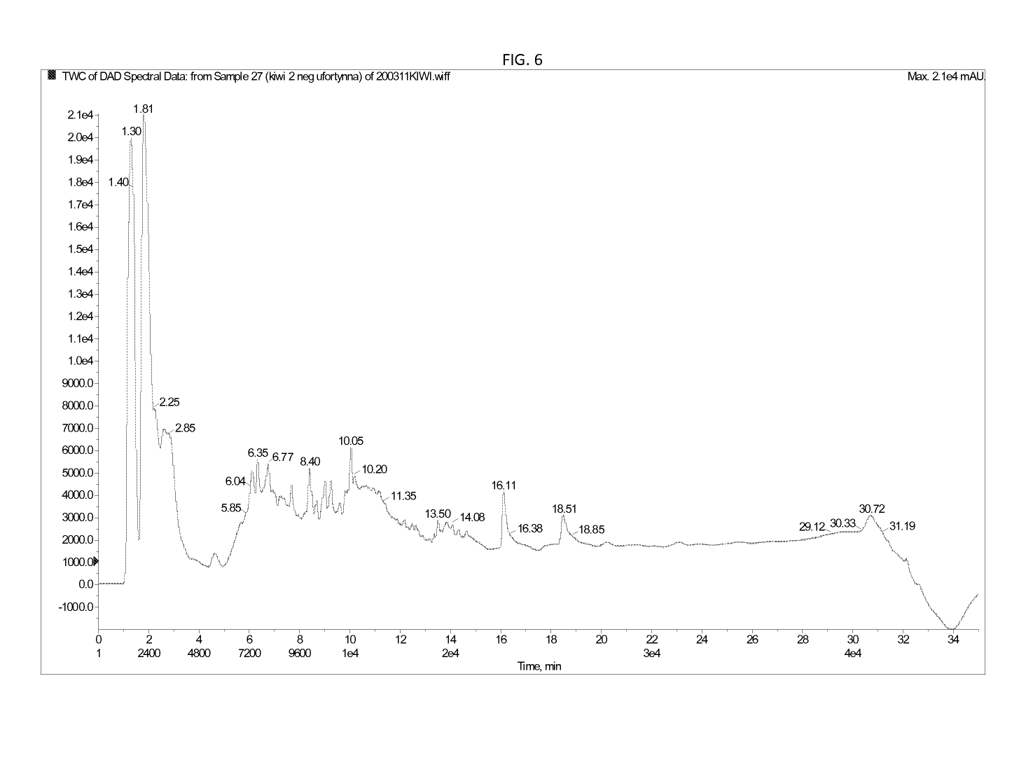

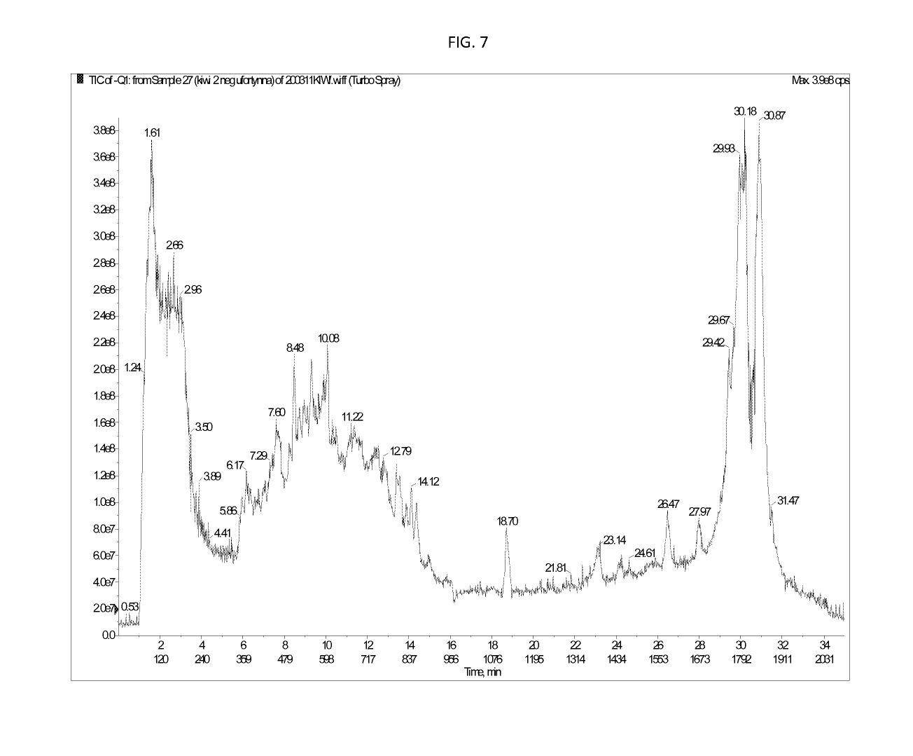

The kiwifruit juice was prepared and the juice was clarified by centrifugation at 9000 g for 15 min. The supernatant was then boiled at 90 C for 20 min. The cooled extract was then centrifuged again at 9000×g for 15 min. The colorless supernatant was then passed through a LIPDEX-1000 column to remove any associated lipids. The eluted delipidated sample was then freeze dried and passed through the 1000 dalton molecular cut-off filter. The filtrate was then run in triple stage LC-MS/MS-UV. MS scans 100-1000 Mw in negative mode (FIG. 7) and UV spectral 200-400 nm (FIG. 6).

The column is a Zorbax 1.8 μM particle rapid resolution C18 column (4.6 mm×50 mm, 1.8 μm). Elution was accomplished by starting 100% mobile phase (A) water-formic acid (100:0.1, v/v/v) to 100% B acetonitrile-formic acid (100:0.1, v/v/v) during 35 minutes.

### Example 4

This example describes the effect of administration of kiwifruit extract on platelet aggregation in human subjects.

The kiwifruit extract was prepared as described above. The final preparation yield was 4-5 g per 100 g of fruits and that contained 45-50% sugar. 20 gm of KFE was mixed with 200 ml of Tine Milk Orange juice for consumption. Six healthy adults of both sexes were recruited into the study. Subjects were aged 25-60 y and had no history of serious disease or hemostatic disorders. Suitability for inclusion into the study was assessed by using diet and lifestyle questionnaires and by medical screening, during which platelet function was assessed. Subjects were selected on the basis of high platelet function, as determined by the platelet aggregation response to 3 μmol ADP/L. Subjects habitually consuming dietary supplements (e.g., fish oils) were asked to suspend these supplements for a minimum of 1 month before participating in the study. Subjects were instructed to abstain from consuming drugs known to affect platelet function for a 10-d period before participation.

Written informed consent was obtained from all subjects. This study was approved by the Oslo authority. Volunteers were overnight fasted. Venous blood samples of ≈20 mL were drawn at each sampling time point (time 0) and then they were asked to drink 200 ml orange juice containing 20 g KFE. For measurements of platelet function blood was collected into plastic syringes and transferred into citrated blood collection tubes (final sodium citrate concentration, 13 mmol/L).

Ex Vivo Platelet Aggregation Studies.

Measurement of the extent of ADP-induced platelet aggregation in PRP was carried out at each time point. The platelet response to suboptimal ADP concentrations was also of interest; under these conditions, a biphasic aggregation response may be observed, which provides further information about the nature of the platelet response. A standardized ADP concentration (3 μmol/L) was used for all measurements. For ex vivo studies, effects on platelet aggregation observed after treatment or control interventions are expressed as the percentage change in area under the aggregation curve after consumption of extract or placebo, as compared with baseline values.

TABLE 3
The results are as here expressed as % inhibition

| Volunteers | % inhibition after 2 hours |
|---|---|
| 1 | 8.1 |
| 2 | 12.6 |
| 3 | 12 |
| 4 | 15 |
| 5 | 8.2 |

### Example 5

This example describes removal of water soluble sugars from free kiwifruit extract using solid phase extraction column chromatography

Kiwifruit Extract Preparation.

Four kiwifruits (1 class, New Zealand) weighing 476.34 g were peeled and weighed again (388.12 g). The peeled flesh was homogenised using the Brown Turbo Mixer ca. 20-30 s, and the whole homogenate was boiled at 96 C for 20 minutes The boiled fruit homogenate was weighted (311.09 gr), and then centrifuged at 13000×g for 30 minutes at room temp. The supernatant was then collected and dried by lyophilzation. The weight of the dried powder was 19.65 g. This fraction was termed as KFE-0 (kiwifruit extract-0).

Solid phase extraction column chromatography for removal of sugars from the KFE-O was performed using the Bond Elut ENV cartridge (Agilent). This column is routinely used to remove the water-soluble sugars from fruit extracts.

The Bond Elut ENV cartridges were conditioned with 2×4 ml 100% Methanol, and then equilibrated with 2×4 ml distilled water. 0.8 g of KFE-0 was dissolved in 4 ml of the distilled H₂O and loaded onto this cartridge. The cartridges were then washed with 2×3 ml distilled water, and the water soluble component was eluted by water. The cartridges were then dried out completely before elution of the non-sugar components in the materials.

The non-sugar components were then eluted with 3×2 ml 100% Methanol under slow (drop wise) flow rates and the eluates were collected into tubes. The eluted samples were evaporated to dryness under N₂ at 45° C., and then recovered in milliQ water. Both the water-eluates and methanol-eluates were then used for their inhibitory activities against platelet aggregation. The yield of the non-sugar component was 0.41 g from 19.6 g of lyophilized materials.

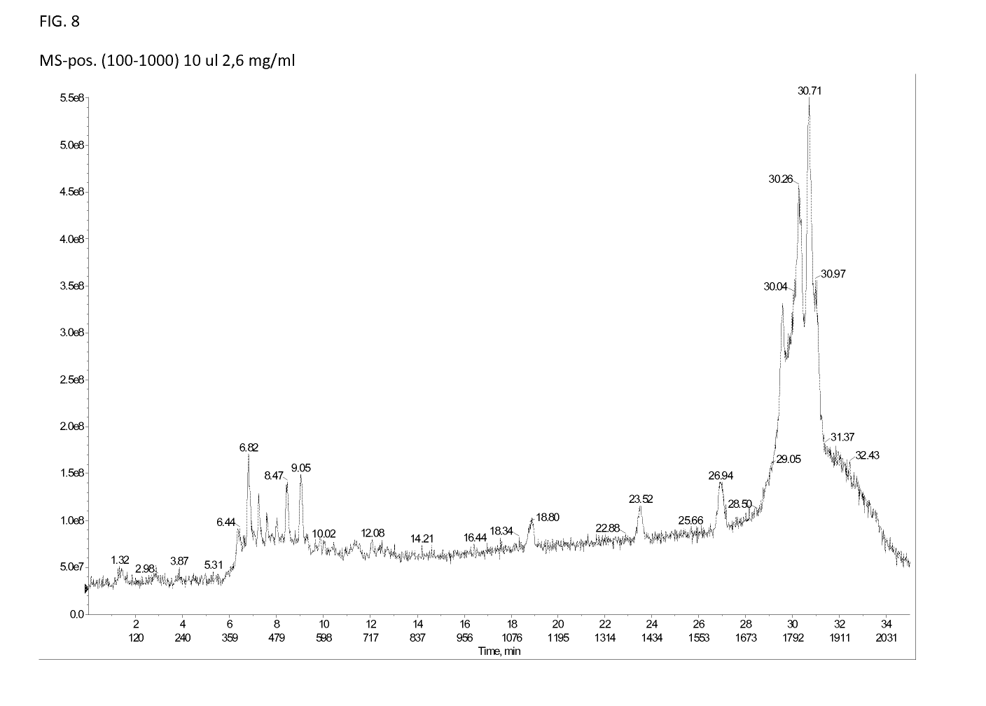

UV and MS spectra of the extract were prepared as in Example 3. The results are presented in FIGS. 8, 9 and 10.

Platelet Aggregation Tests on PRP by Use of Helena AggRAM Aggregometer.

The pH of all samples was adjusted to 7.4 prior to testing their effect on platelet aggregation.

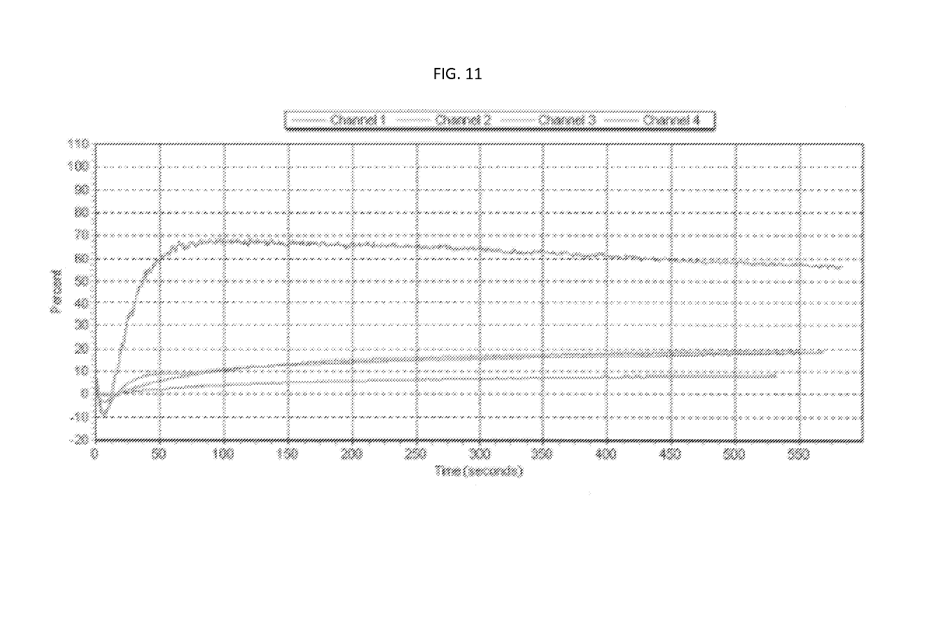

Inhibitory effect of the eluates was determined by pre-incubating 225 μl of platelet rich plasma (PRP) with 25 μl of eluates (water soluble eluates and methanol eluates) for 30 minutes. Platelet aggregation was then tested by adding 25 μl of 5 μM ADP or 25 μl of different concentrations of collagen (1-10 μg/ml) in PRP. The methanolic eluates showed a strong and dose dependent inhibition against both ADP and collagen-induced platelet aggregation whereas water-eluted sugar components had no effects. FIG. 11 shows the strong inhibitory effect of methanolic eluates at different concentrations on ADP-induced platelet aggregation. This clearly demonstrates that the anti-platelet components are sugar-free components of kiwifruit.

### Example 6

This example describes the calculation of the amount of sugar free active components in the fruits. 0.30 g of active sugar free materials were isolated from 19.6 g of the lyophilized material, and since 19.6 g of lyophilized material was obtained from 476 grams of fruit, this indicates that the active sugar free components present in the amounts of 0.063 gms per 100 g of whole kiwifruit.

### Example 7

This example demonstrates inhibition of plasma Angiotensin converting enzyme (ACE) activity by the kiwifruit extract. The effect of KFE-0 on the human plasma ACE activity was measured by incubating the plasma (250 μl) with 25 μl of KFE using kit by Bhulmann. KFE (25 μl) significantly inhibited the ACE activity by 15% compared with control (25.1+/−2.1 U/L versus 29.5+/−5.0 U/L; n=5.

### Example 8

This example demonstrates reduction of blood pressure following administration of kiwi fruit to male smokers.

### Materials and Methods

Subjects.

Participants were recruited through advertisement in local newspapers. Inclusion criteria were male, aged 45-75 years, smoking 45 cigarettes per day, stable weight range (<4-kg change last 12 weeks) and body mass index (<35 kg m⁻²). The exclusion criteria were any history of CVD or other significant clinical disorders, following a vegetarian or near-vegetarian diet, or allergy to foods included in the intervention diets. We excluded subjects with a history of serious or unstable medical or psychiatric disorder; current use of lipid-lowering treatment, aspirin or non-steroidal anti-inflammatory drugs; nutritional supplements or herbs for weight loss; or participants in drug trials during the previous 30 days. Of the 102 study subjects, 8 reported ongoing use of BP-lowering agents (angiotensin-II receptor antagonists, ACE inhibitors, calcium antagonist and b-blockers). Use of drugs was stable through the run-in and intervention periods.

Study Design and Intervention.

The study followed a randomized, parallel design with an 8-week intervention period, and a 4-week run-in period preceded the intervention period. During the run-in and intervention periods, participants were instructed to avoid use of vitamin or antioxidant supplementation, as well as pain or cold remedies containing aspirin. Moreover, participants were asked to limit their consumption of berries, nuts, pomegranates, tomatoes, kiwifruit, tea and coffee (<3 cups per day). After the run-in period, subjects were randomly assigned to one of three groups, kiwifruit, antioxidant-rich diet or control group.

The kiwifruit group received 3 kiwifruits per day (Actinidia deliciosa; Odd Langdalen Frukt & Grønnsaker Engros A S, Oslo, Norway). This provided an addition of approximately 195 g fruit per day, providing 467 kJ day⁻¹. Subjects in both intervention groups were provided with intervention items at weekly follow-ups. For the antioxidant-rich diet group, the administered food items, as well as the amounts of dietary antioxidants provided, are specified in Table 4. To participants in the antioxidant-rich diet group, individual counseling was given by a trained nutritionist to help implement the provided foods in their habitual diet. The control group was advised to follow their habitual diet, and attended bi-weekly follow-ups. The study was approved by the regional ethics for medical research committee (REK Sør) and all participants gave written, informed consent. The study is registered as ‘Oslo antioxidant study’ (NCT00520819) at clinicaltrials.gov.

Dietary Intake.

Dietary intake was recorded using a 7-day food record with a picture book. Previous validation of this food record has demonstrated that reported energy and nutrient intakes are valid on a group level. 14 The food records were completed the last week of the run-in period and the last week of the intervention period. Average daily intakes were computed using the food database AE-07 and the KBS software system (version 4.9, 2008) developed at the Department of Nutrition. The food database is based on the 2006 edition of the Norwegian food composition table. The antioxidant values are based on our comprehensive antioxidant food database. 10 Data on dietary intakes were available for 32 participants in each group.

BP Measurements.

BP was measured by a trained nurse using a digital BP monitor (OMRONHem-705 CP, Kyoto, Japan) and appropriate cuff sizes after 5-min rest. BP was calculated as the mean of three measurements. BP was classified according to the ‘2007 Guidelines for the Management of Arterial Hypertension’ of the European Society for Hypertension. 15 Accordingly, subjects were classified as ‘optimal’ if systolic BP was <120 mmHg or diastolic BP was o80 mmHg; ‘normal/high normal’ if systolic BP was 120-139 mmHg and/or diastolic BP was 80-89 mmHg; and ‘hypertensive’ if systolic BP was X140 mmHg and/or diastolic BP was X90 mmHg.

Laboratory Analysis.

Overnight, fasting blood samples were collected before and after the intervention period, between 0800 and 1000 hours. Plasma and serum were immediately prepared and stored at −70° C. until the time of analysis unless immediately analyzed. The methods for assessment of serum lipids, enzyme activities, and inflammatory and hemostatic parameters related to CVD are described elsewhere.

Platelet Aggregation and ACE Activity.

Adenosine diphosphate (ADP)-induced whole-blood platelet aggregation was assessed in citrated whole blood using a platelet aggregometer (Chrono-Log, Haverton, Pa., USA) at a constant stirring speed of 1000 r.p.m. at 37° C. as described elsewhere. 7 Based on previous experiments, and the high number of samples assessed each day, ADP at 5 mM was the only agonist used. 7 Platelet aggregation was assessed within 2 h after blood sampling, and samples were kept at room temperature until the time of analysis. ACE activity was assessed in serum by its ability to cleave the synthetic substrate (FAPGG). The assays linear range is between 0 and 175 UI⁻¹. ACE activity was determined in thawed serum according to the manufacturer's instructions (ACE kinetic, kit number 01-KK-ACK; Buuhlmann Laboratories AG, Schonenbuch, Switzerland). These assays have an interassay coeffecients of variation <7%.

Statistical Analysis.

Kruskal-Wallis one-way analysis of variance was used to compare baseline values or changes during the intervention period between groups. Changes (that is, intervention effects) were calculated by subtracting the baseline value from the post-intervention value. Where significant results were obtained, Student's t-test or Mann-Whitney non-parametric test was used to compare differences. Data from two excluded participants were not included in the analysis. All statistics were performed using SPSS version 18.0. P-value o0.050 was considered statistically significant.

### Results

A total of 137 subjects responded to the initial advertisement, and 118 were found eligible and screened. Of these, 102 were included in the study. Two subjects were excluded: one subject in the kiwifruit group owing to a non-fatal cardiovascular event and one in the antioxidant-rich diet group owing to lack of compliance. A total of 100 participants completed the study (n=34 in the control group, n=33 in each intervention group; Data not shown).\

Baseline characteristics were similar between groups (Table 5), and no change in body mass index, body weight or cigarette smoking was observed during the intervention period. Compliance to both intervention diets was good. Dietary intakes at baseline and changes during the intervention period are listed in Table 6. Dietary intake was similar between groups at baseline. Baseline levels, as well as the effects of the dietary interventions on BP, are given in Table 7, for the overall study population, and among subjects with optimal, normal/high-normal BP, and hypertensives. In the kiwifruit group, reductions of 10 mmHg in systolic BP and 9 mmHg in diastolic BP (P=0.007 and P=0.010; change from baseline in the kiwifruit group compared with change from baseline in the control group) were observed in the overall study population. The largest effects were observed among subjects with normal/high-normal BP and hypertensives. Interestingly, a reduction of 10 mmHg in systolic BP was also observed among hypertensives in the antioxidant-rich diet group (P=0.045). Furthermore, we observed that the number of subjects with normal/high-normal BP, or hypertensives, in the kiwifruit group was significantly reduced, from 65% at baseline to 33% following the intervention (P<0.050; data not shown). Only minor changes were observed in the antioxidant rich-diet group. Effects of the dietary interventions on platelet aggregation and ACE activity are presented in Table 8. In the kiwifruit group, a 15% reduction in platelet aggregation (P<0.004) and an 11% reduction in ACE activity (P=0.013) were observed. No similar effects were observed in the antioxidant-rich diet group.

No effects were observed on plasma lipids, other enzyme activities, and inflammatory and hemostatic parameters related to CVD (Supplementary Table ST 1).

These data demonstrate that intake of 3 kiwifruits per day promotes pronounced anti-hypertensive and anti-thrombotic effects in male smokers. Additionally, a substantial reduction in systolic BP among hypertensives following consumption of the antioxidant-rich diet was observed.

TABLE 4
Food items provided by the antioxidant rich diet.

| | | | Total | |
|---|---|---|---|---|
| | | | antioxidant | Energy |
| | Provided | | (mmol | (kJ |
| Food item | weekly | | per week) | per week) |
| Twinings Green Java Tea | 7 | tea bags | 20.86 | 45 |
| Juice of rose lips, orange, | 1.66 | l | 42.64 | 1397 |
| apple and carrot (Mona | | | | |
| Yellow)^{a} | | | | |
| Juice of carberries, raspberries | 1.66 | l | 10.93 | 1397 |
| and grapes (Mona Red)^{a} | | | | |
| Juice of block chokeberry, | 1.66 | l | 23.18 | 1397 |
| biltorries, grapes and cherries | | | | |
| (Mona Blue)^{a} | | | | |
| Bilberry juice (Vaccinium^{b} | 0.66 | l | 54.73 | 1069 |
| Bilberry jam (V. myrtillus)^{c} | 345 | g | 10.50 | 565 |
| Bilberries (V. myrtillus)^{d} | 200 | g | 16.24 | 312 |
| Blackberries (Rubus | 200 | g | 9.20 | 288 |
| fructoosus)^{d} | | | | |
| Strawberries (Fragaria x | 200 | g | 4.26 | 321 |
| ananossa)^{d} | | | | |
| Raspberries (Rubus idoeus)^{d} | 200 | g | 5.87 | 169 |
| Pomegranate (Punica | 200 | g | 3.43 | 608 |
| granatum)^{d} | | | | |
| Dark blue grapes (Viris sp.)^{d} | 200 | g | 2.23 | 284 |
| Brussel sprouts (Brassica | 200 | g | 2.01 | 288 |
| olerocea var. gemmifero)^{d} | | | | |
| Broccoli (Brassica olerocea | 200 | g | 1.93 | 189 |
| var. italica)^{d} | | | | |
| Red cabbage Oleracea var. | 200 | g | 9.82 | 189 |
| capitata rubra), | | | | |
| outdoor cultivar^{e} | | | | |
| Kale (Brassica oleracea var. | 200 | g | 4.71 | 345 |
| sabellica)^{e} | | | | |
| Blue potatoes (Solanum | 150 | g | 1.20 | 419 |
| tuberosum, ‘Blue cargo’)^{d} | | | | |
| Tomatoes (Solanum | 700 | g | 2.24 | 822 |
| lycopersicum)^{d} | | | | |
| Dark chocolate, 70% cocoa^{f} | 100 | g | 11.22 | 1850 |
| Pecan nuts (Carya | 100 | g | 8.30 | 2787 |
| illonoinensis)^{g,h} | | | | |
| Sunflower seeds (Helianthus | 100 | g | 6.31 | 2269 |
| anniuus)^{g,h} | | | | |
| Walnuts (Juglans californica)^{i,e} | 200 | g | 44.48 | 5657 |
| Eara Virgin Olive Oil | 0.003 | l | 0.19 | 2302 |
| (Olea europaea)^{j} | | | | |
| Rosemary (Rosmarinus | 3 | g | 1.55 | 0 |
| officinalis)^{k} | | | | |
| Thyme (Thymus vulgaris)^{k} | 3 | g | 1.69 | 0 |
| Oregano (Origanum vulgare | 3 | g | 1.90 | 0 |
| gracile)^{k} | | | | |
| In total/week | | | 305.60 | 24857 |

Food items were provided by
^{a}Tine BA (Osla, Norway), or purchased from the following:
^{b}Corona Safteri (Ranheim, Norway).
^{c}Heistad (Bergen, Norway),
^{d}Odd Langdalen Engros (Oslo, Norway),
^{e}The Norwegian University of Life Sciences (Äs, Norway),
^{f}Kraft Foods (Northfield, IL., USA),
^{g}Den Iille nettefrabrikken (Fredrikstad, Norway).
^{h}Provided as ingredients in bread (Äpent bakeri, Oslo, Norway).
ⁱDiamond Foods Inc (Stockton, CA, USA).
^{j}Ybarra (Toano, Spain),
^{k} Black Boy (Bergen, Norway).
Amounts of total antioxidants were accessed by FRAP assay.¹⁰

TABLE 5
Baseline descriptives

| | | | Antioxidant- | | | | |
|---|---|---|---|---|---|---|---|
| | | | rich diet | | | | |
| | Control (n = 34) | | (n = 33) | | Kiwifruit (n = 33) | | |
| Variable | Baseline (target) | | Baseline (target) | | Baseline (target) | | P* |
| Age (years) | 56 | (44-71) | 57 | (45-74) | 57 | (45-71) | 0.770 |
| BMI (kg m⁻²) | 24.8 | (21.6-30.4) | 25.7 | (19.4-32.0) | 24.7 | (15.8-32.2) | 0.897 |
| Body weight (kg) | 83.7 | (56.2-93.8) | 70.3 | (50.7-1.10) | 76.8 | (55.0-98.2) | 0.270 |
| Cigarettes per day | 15 | (6-35) | 15 | (5-40) | 15 | (5-25) | 0.987 |

| | | | | |
|---|---|---|---|---|
| BP-lowering agents (n) | 3 | 2 | 3 | 1.000 |

*P-value is from comparing baseline values between groups.

TABLE 6
Daily intake of macro- and micronutrients, baseline and change during intervention ^{a, b}

| | Control (n = 32) | | | Antioxidant-rich diet (n = 32) | | | Kiwifruit (n = 32) | | | |
|---|---|---|---|---|---|---|---|---|---|---|
| Variable | Baseline (range) | | Change | Baseline (range) | | Change | Baseline (range) | | Change | P |
| Total energy | 9787 | (4945-16780) | −519 ± 2213 | 10,282 | (5334-16857) | 1957* ± 2350 | 10,001 | (5225-19,638) | −18.5* ± 1961 | <0.001 |
| (kJ) | | | | | | | | | | |
| Protein | 16.3 | (12.1-22.0) | −0.40 ± 2.0 | 16.1 | (11.2-23.1) | −2.8* ± 2.7 | 16.6 | (12.9-23.5) | −1.0* ± 2.53 | 0.001 |
| (E %) | | | | | | | | | | |
| Total fat | 34.4 | (23.7-48.9) | −0.02 ± 4.7 | 34.5 | (25.5-53.9) | 0.9 ± 5.7 | 35.3 | (25.8-45.5) | −0.35 ± 4.50 | 0.671 |
| (E %) | | | | | | | | | | |
| Carbo- | 42.2 | (24.1-56.8) | −5.0 ± 7.4 | 41.2 | (28.6-50.9) | 4.5* ± 4.8 | 40.6 | (31.2-57) | 1.96* ± 4.32 | 0.001 |
| hydrates | | | | | | | | | | |
| (E %) | | | | | | | | | | |
| Fiber (g) | 22.5 | (18.9-36.0) | −2.8 ± 6.6 | 20.2 | (18.0-22.5) | 17.3* ± 19.3 | 22.9 | (20.3-25.6) | 3.7* ± 4.00 | <0.001 |
| Cholesterol | 370 | (187-677) | −20 ± 165 | 375 | (204-603) | −29 ± 184 | 378 | (156-878) | −6 ± 137 | 0.226 |
| (mg) | | | | | | | | | | |
| Calcium | 515 | (237-1822) | −47 ± 263 | 834 | (254-1597) | 85 ± 386 | 792 | (485-1810) | −17 ± 267 | 0.236 |
| (mg) | | | | | | | | | | |
| Magnesium | 402 | (214-961) | −22 ± 90 | 403 | (237-608) | 124* ± 119 | 390 | (207-700) | 40.8* ± 68.2 | <0.001 |
| (mg) | | | | | | | | | | |
| Potassium | 4020 | (2020-6107) | −208 ± 884 | 4077 | (2179-5920) | 1187* ± 1324 | 4008 | (1837-5348) | 236* ± 704 | 0.002 |
| (mg) | | | | | | | | | | |

*Significantly different as compared with control (P < 0.050).
^{a}Based on 7-day food record.
^{b}All variables are normally distributed.

TABLE 7
baseline values (Range) and change in BP during the intervention; in the overall study population,
and among subjects with optimal BP, normal/high-normal BP and hypertensives^{a}.

| | Control (n = 34) | | | | Antioxidant-rich diet (n = 33) | | | | Kiwifruit (n = 33) | | | | |
|---|---|---|---|---|---|---|---|---|---|---|---|---|---|
| Variable | Baseline (range) | | Change | | Baseline (range) | | Change | | Baseline (range) | | Change | | P |
| Overall study population | 34 | | | | 13 | | | | 33 | | | | |
| Systolic BP (mmHg) | 123 | (108-187) | 2 ± 11.1 | | 127 | (98-160) | −2 ± 10.7 | | 126 | (89-175) | −10* ± 11 | | 0.019 |
| Diastolic BP (mmHg) | 83 | (70-102) | −2 ± 8.8 | | 83 | (67-113) | −4 ± 7.4 | | 86 | (64-110) | −9* ± 11 | | 0.015 |
| Subjects with optimal BP | 11 | | | | 11 | | | | 12 | | | | |
| Systolic BP (mmHg) | 112 | (108-117) | 9 | (2, 16) | 109 | (98-118) | 3 | (−4, 9) | 111 | (89-119) | −1 | (−6, 4) | 0.011 |
| Diastolic BP (mmHg) | 75 | (70-79) | 4 | (−3, 11) | 76 | (67-78) | −1* | (−4, 3) | 74 | (64-79) | −3* | (−7, 0) | 0.006 |
| Subjects with normal/ | 12 | | | | 13 | | | | 12 | | | | |
| high-normal BP | | | | | | | | | | | | | |
| Systolic BP (mmHg) | 127 | (120-135) | −3 | (−9, 3) | 129 | (120-139) | −2 | (−9, 4) | 127 | (120-132) | −13 | (−19, −7) | 0.045 |
| Diastolic BP (mmHg) | 82 | (80-89) | −2 | (−8, 3) | 82 | (80-88) | −5 | (−11, −1) | 84 | (80-89) | −8* | (−14, −4) | 0.049 |
| Hypertensive | 13 | | | | 9 | | | | 9 | | | | |
| Systolic BP (mmHg) | 153 | (140-187) | −1 | (−7, 5) | 149 | (140-160) | −10* | (−17, −2) | 153 | (140-175) | −15* | (−30, −2) | 0.041 |
| Diastolic BP (mmHg) | 98 | (91-102) | −4 | (−11, 3) | 94 | (92-113) | −5 | (−11, 2) | 99 | (90-110) | −13* | (−20, −5) | 0.043 |

Abbreviation: BP, blood pressure.
^{a}Significantly different from change in the contol group (P < 0.050, Mann-Whitney test).
^{b}BP is non-normally distributed in the overall study population and within sub-groups.

TABLE 8
Baseline values (range), and change in whole-blood platelet aggregation and ACE activity^{a}.

| | Control (n = 34) | | | | Antioxidant-rich diet (n = 33) | | | |
|---|---|---|---|---|---|---|---|---|
| Variable | Baseline | | Change | | Baseline | | Change | |
| Aggregation (AU) | 13.6 | (10.0-16.5) | −0.5 | (−1.3, 0.1) | 13.0 | (10.0-16.3) | −1.1 | (−1.8, −0.3) |
| ACE activity (UI⁻¹) | 27.42 | (6.89-59.23) | 3.09 | (−0.95, 6.05) | 27.31 | (15.5-62.73) | −1.4 | (−3.20, 4.15) |

| | Kiwifruit (n = 33) | | | | |
|---|---|---|---|---|---|
| Variable | Baseline | | Change | | P |
| Aggregation (AU) | 13.6 | (10.0-18.3) | −2.1* | (−3.1, −1.4) | 0.009 |
| ACE activity (UI⁻¹) | 28.45 | (11.84-53.20) | −3.22* | (−5.80, −1.15) | 0.034 |

Abbreviation: ACE, angiotensin-converting enzyme.
^{a}Significantly different from change in the contol (P < 0.050, Mann-Whitney test).
^{b}Variable are non-normally distributed baseline is median (range); change is median (9.3% CI).

### Example 9

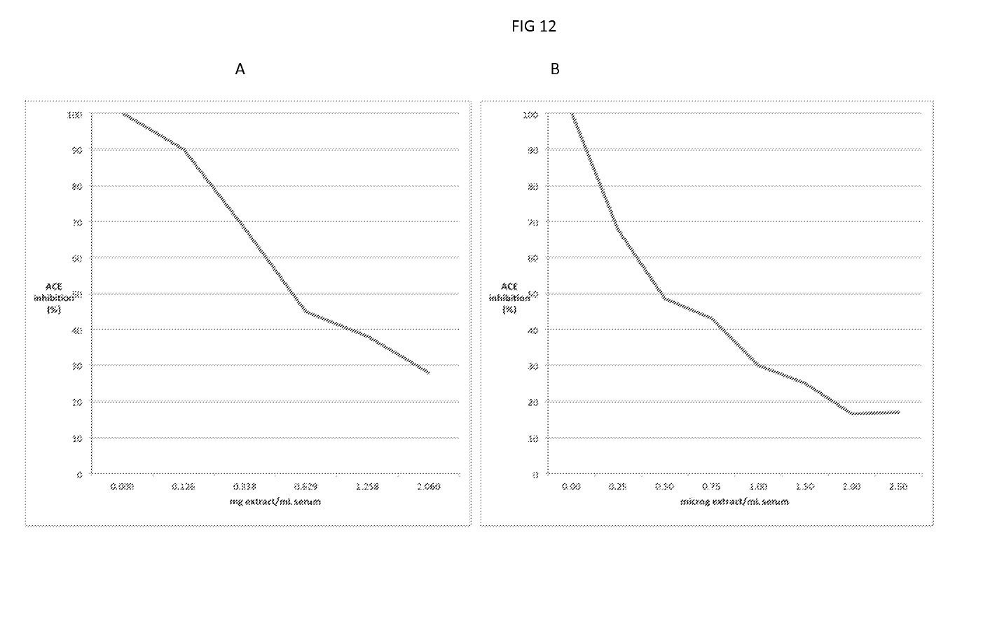

This example provides a comparison of the ACE activity of the sugar free kiwi fruit extract (KFE) and a synthetic ACE inhibitor captopril. The results are presented in FIG. 12. As can be seen, the KFE exhibits a similar dose response curve as the synthetic agent, although approximately 1000-fold more of the KFE is required. Even though the higher amount is required, this amount is within a normal nutraceutical dosing range and it is surprising that this amount of ACE inhibitory activity can be achieved with a naturally occurring substance.

### Example 10

This example provides a summary of ACE activity through the various process steps. Table 9 provides the AE activity of fresh kiwi fruit juice. Table 10 provides the ACE activity of boiled kiwi fruit juice. Table 11 provides the ACE activity of sugar free kiwi fruit extract. Table 12 provides a summary of the mass balanced adjusted ACE activity for the extracts at the different stages of processing. As can be seen, the ACE activity is present through the processing steps at similar potency when adjusting for mass-balance.

TABLE 9
ACE activity of KFJ (fresh juice)

| Kiwifruit juice | |
|---|---|
| (dry weight mg/ml) | % ACE activity |
| 0 | 96 |
| 32 | 68 |
| 64 | 58 |

TABLE 10
ACE activity of KFJb (boiled juice)

| Kiwifruit juice | |
|---|---|
| (dry weight mg/ml) | % ACE activity |
| 0 | 100 |
| 4 | 81 |
| 8 | 77 |
| 12 | 74 |
| 15 | 68 |
| 19 | 65 |

TABLE 11
ACE activity of KFE (sugar free extract)

| KFE | ACE |
|---|---|
| mg/ml | % Activity |
| 0.000 | 100 |
| 0.126 | 90 |
| 0.338 | 68 |
| 0.629 | 45 |
| 1.258 | 38 |
| 2.060 | 28 |

TABLE 12
Mass balance adjusted ACE activity:
(IC-32; 32% inhibition of ACE activity)

| | | Mass balance | |
|---|---|---|---|
| Processing steps: | IC-32 | (dry) | Relative potency |
| KFJ | 32 mg/ml | 6 gr | 100 |
| KFJb | 15 mg/ml | na | na |
| KFE | 0.338 mg/ml | .07 gr | 110 |

### Example 11

This example provides a comparison of ACE inhibitory activity of kiwi fruit extract and orange fruit extract prepared using the sugar free extract procedure described above. The potency of a kiwi fruit extract is about 300 fold higher than an orange extract, using identical processing steps as for the kiwi extract. See Tables 13 and 14.

TABLE 13
ACE activity of kiwi fruit extract (KFE; sugar free extract)
Sugar free KFE extract

| KFE | ACE |
|---|---|
| mg/ml | % Activity |
| 0.000 | 100 |
| 0.126 | 90 |
| 0.338 | 68 |
| 0.629 | 45 |
| 1.258 | 38 |
| 2.060 | 28 |

TABLE 14
ACE activity of orange fruit extract (OFE; sugar free extract)
Sugar free OFE extract

| KFE | ACE |
|---|---|
| mg/ml | % Activity |
| 0 | 96 |
| 48 | 87 |
| 96 | 80 |
| 144 | 74 |

### REFERENCES

1. Van H L, McCoin M, Kris-Etherton P M, Burke F, Carson J A, Champagne C M et al. The evidence for dietary prevention and treatment of cardiovascular disease. J Am Diet Assoc 2008; 108(2): 287-331.
2. Willcox J K, Catignani G L, Lazarus S. Tomatoes and cardiovascular health. Crit Rev Food Sci Nutr 2003; 43(1): 1-18.
3. Dohadwala M M, Vita J A. Grapes and cardiovascular disease. J Nutr 2009; 139(9): 1788S-1793S.
4. Erlund I, Koli R, Alfthan G, Marniemi J, Puukka P, Mustonen P et al. Favorable effects of berry consumption on platelet function, blood pressure, and HDL cholesterol. Am J Clin Nutr 2008; 87(2): 323-331.
5. Ried K, Sullivan T, Fakler P, Frank O R, Stocks N P. Does chocolate reduce blood pressure? A meta-analysis. BMC Med 2010; 8(1): 39.
6. Chang W H, Liu J F. Effects of kiwifruit consumption on serum lipid profiles and antioxidative status in hyperlipidemic subjects. Int J Food Sci Nutr 2009; 19: 1-8.
7. Duttaroy A K, Jorgensen A. Effects of kiwi fruit consumption on platelet aggregation and plasma lipids in healthy human volunteers. Platelets 2004; 15(5): 287-292.
8. Tandon R, Sinha M K, Garg H, Khanna R, Khanna H D. Oxidative stress in patients with essential hypertension. Natl Med J India 2005; 18(6): 297-299.
9. Bjelakovic G, Nikolova D, Gluud L L, Simonetti R G, Gluud C Antioxidant supplements for prevention of mortality in healthy participants and patients with various diseases. Cochrane Database Syst Rev 2008; (2). CD007176.
10. Carlsen M H, Halvorsen B L, Holte K, Bohn S K, Dragland S, Sampson L et al. The total antioxidant content of more than 3100 foods, beverages, spices, herbs and supplements used worldwide. Nutr J 2010; 9: 3.
11. Karlsen A, Svendsen M, Seljeflot I, Sommernes M A, Sexton J, Brevik A et al. Compliance, tolerability and safety of two antioxidant-rich diets: a randomised controlled trial in male smokers. Br J Nutr 2011; 106(4): 557-571.
12. Chavez J, Cano C, Souki A, Bermu'dez V, Medina M, Ciszek A et al. Effect of cigarette smoking on the oxidant/antioxidant balance in healthy subjects. Am J Ther 2007; 14(2): 189-193.
13. Virdis A, Giannarelli C, Neves M F, Taddei S, Ghiadoni L. Cigarette smoking and hypertension. Curr Pharm Des 2010; 16(23): 2518-2525.
14. Lillegaard I T, Andersen L F. Validation of a pre-coded food diary with energy expenditure, comparison of under-reporters v. acceptable reporters. Br J Nutr 2005; 94(6): 998-1003.
15. Mancia G, De Backer G, Dominiczak A, Cifkova R, Fagard R, Germano G et al. 2007 Guidelines for the management of arterial hypertension: the Task Force for the Management of Arterial Hypertension of the European Society of Hypertension (ESH) and of the European Society of Cardiology (ESC). Eur Heart J 2007; 28(12): 1462-1536.
16. Law M R, Morris J K, Wald N J. Use of blood pressure lowering drugs in the prevention of cardiovascular disease: meta-analysis of 147 randomised trials in the context of expectations from prospective epidemiological studies. BMJ 2009; 338: b1665.
17. Chen C H, Lin J Y, Lin C N, Hsu S Y. Inhibition of angiotensin-I-converting enzyme by tetrahydroxyxanthones isolated from Tripterospermum lanceolatum. J Nat Prod 1992; 55(5): 691-695.
18. Jung K A, Song T C, Han D, Kim I H, Kim Y E, Lee C H. Cardiovascular protective properties of kiwifruit extracts in vitro. Biol Pharm Bull 2005; 28(9): 1782-1785.
19. Lacaille D, Franck U, Wagner H. Search for potential angiotensin converting enzyme (ACE)-inhibitors from plants. Phytomedicine 2001; 8(1): 47-52.
20. Persson I A, Persson K, Andersson R G. Effect of Vaccinium myrtillus and its polyphenols on angiotensin-converting enzyme activity in human endothelial cells. J Agric Food Chem 2009; 57(11): 4626-4629.
21. Heran B S, Wong M M, Heran I K, Wright J M. Blood pressure lowering efficacy of angiotensin converting enzyme (ACE) inhibitors for primary hypertension. Cochrane Database Syst Rev 2008; (4): CD003823.
22. Park Y S, Jung S T, Kang S G, Drzewiecki J, Namiesnik J, Haruenkit R et al. In vitro studies of polyphenols, antioxidants and other dietary indices in kiwifruit (Actinidia deliciosa). Int J Food Sci Nutr 2006; 57(1-2): 107-122.
23. Szeto Y T, Tomlinson B, Benzie I F. Total antioxidant and ascorbic acid content of fresh fruits and vegetables: implications for dietary planning and food preservation. Br J Nutr 2002; 87(1): 55-59.
24. Angiolillo D J, Guzman L A, Bass T A. Current antiplatelet therapies: benefits and limitations. Am Heart J 2008; 156(2 Suppl): S3-S9.
25. Skowasch D, Viktor A, Schneider-Schmitt M, Luderitz B, Nickenig G, Bauriedel G. Differential antiplatelet effects of angiotensin converting enzyme inhibitors: comparison of ex vivo platelet aggregation in cardiovascular patients with ramipril, captopril and enalapril. Clin Res Cardiol 2006; 95(4):
26. Dutta-Roy A K. (2002) Dietary components and human platelet activity. Platelets. 13(2):67-75
27. Dutta-Roy, A K, Crossbi, L, and Gordon, M. J. (2001) Effects of tomato extract on human platelet aggregation in vitro, Platelets, 12(4):218-27.
28. Nishiyama I. Fruits of the actinidia genus. Adv Food Nutr Res. 2007; 52:293-324.
29. Sun-Waterhouse D, Chen J, Chuah C, Wibisono R, Melton L D, Laing W, Ferguson L R, Skinner M A (2009). Kiwifruit-based polyphenols and related antioxidants for functional foods: kiwifruit extract-enhanced gluten-free bread. Int J Food Sci Nutr. 60 Suppl 7:251-64. Epub
30. Daigo Abe, Takeshi Saito, Yasutaka Kubo, Yoshimasa Nakamura, Keizo Sekiya A fraction of unripe kiwi fruit extract regulates adipocyte differentiation and function in 3T3-L1 cells, Biofactor vol.

All publications and patents mentioned in the above specification are herein incorporated by reference. Various modifications and variations of the described method and system of the invention will be apparent to those skilled in the art without departing from the scope and spirit of the invention. Although the invention has been described in connection with specific preferred embodiments, it should be understood that the invention as claimed should not be unduly limited to such specific embodiments. Indeed, various modifications of the described modes for carrying out the invention that are obvious to those skilled in the field of this invention are intended to be within the scope of the following claims.

1. A composition comprising a fruit extract from a fruit of the family Actinidia, said extract characterized as being de-sugared and having a biological activity.

2. The composition of claim 1, wherein extract comprises less than about 30% w/w sugars.

3-5. (canceled)

6. The composition of claim 1, wherein said extract is characterized in being substantially free of sugars.

7. The composition of claim 1, wherein said composition is stable.

8. The composition of claim 1, wherein said composition retains biological activity during storage.

9. The composition of claim 1, wherein said fruit extract is stabilized by a method selected from the group consisting of ultrafiltration, heat treatment, and combinations thereof.

10. The composition of claim 9, wherein said heat treatment comprises heating to at least 90 degrees Celsius.

11. The composition of claim 1, wherein said biological activity is inhibition of platelet aggregation in an in vitro platelet aggregation assay.

12. The composition of claim 1, wherein the extract has more than 4% inhibitory activity in an in vitro platelet aggregation assay after kept at 4 degrees Celsius for 24 days normalized to day 0.

13. The composition of claim 1, further characterized in retaining at least 80% of biological activity of said biologically active molecules when stored for 4 days at 4 degrees Celsius as compared to a fresh extract fraction, wherein said biological activity is inhibition of platelet aggregation in an in vitro platelet aggregation assay.

14. The composition of claim 1, further characterized in retaining at least 80% of biological activity of said biologically active molecules when stored for at least 18 days at 4 degrees Celsius as compared to a fresh extract fraction, wherein said biological activity is inhibition of platelet aggregation in an in vitro platelet aggregation assay.

15. The composition of claim 1, further characterized in retaining at least 80% of biological activity of said biologically active molecules when stored for at least 24 days at 4 degrees Celsius as compared to a fresh extract fraction, wherein said biological activity is inhibition of platelet aggregation in an in vitro platelet aggregation assay.

16. The composition of claim 1, wherein said biological activity is inhibition of angiotensin-converting enzyme.

17. The composition of claim 1, wherein said extract is delipidated.

18. The composition of claim 1, wherein said fraction comprises biologically active molecules with a molecular weight of less than 3000 daltons.

19. The composition of claim 1, wherein said fraction comprises biologically active molecules with a molecular weight of less than 1000 daltons.

20. The composition of claim 1, wherein said fruit extract exhibits major peaks at approximately 8.38 and 9.25 minutes on a UV spectrum scan of liquid chromatography of said extract on a Zorbax 1.8 μM particle rapid resolution C18 column (4.6 mm×50 mm, 1.8 μm) with a 100% mobile phase (A) water-formic acid (100:0.1, v/v/v) to 100% B acetonitrile-formic acid (100:0.1, v/v/v) during 35 minutes.

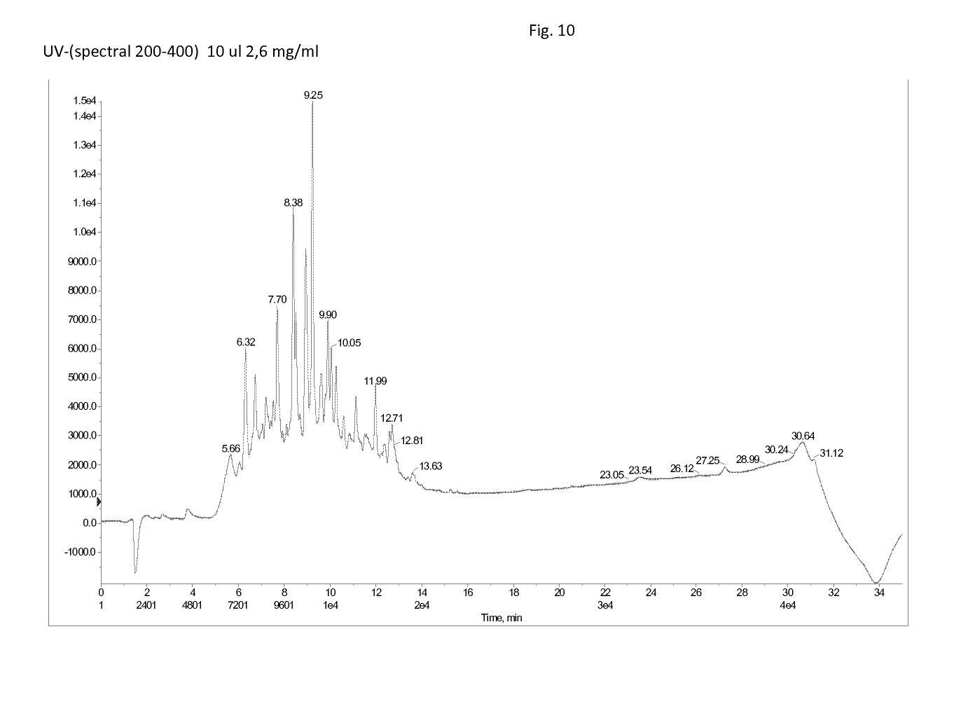

21. The composition of claim 13, wherein said extract exhibits major UV spectrum peaks as observed in FIG. 10.

22. The composition of claim 1, wherein said fruit extract exhibits major peaks at approximately 30.26, and 30.71 in a mass spectometry 100-1000 Mw in positive mode scan of liquid chromatography of said extract on a Zorbax 1.8 μM particle rapid resolution C18 column (4.6 mm×50 mm, 1.8 μm) with a 100% mobile phase (A) water-formic acid (100:0.1, v/v/v) to 100% B acetonitrile-formic acid (100:0.1, v/v/v) during 35 minutes.

23. The composition of claim 22, wherein said extract exhibits major total ion current chromatogram peaks as observed in FIG. 8.

24. The composition of claim 1, wherein said fruit extract exhibits a major peak at approximately 30.79 in a mass spectometry 100-1000 Mw in negative mode scan of liquid chromatography of said extract on a Zorbax 1.8 μM particle rapid resolution C18 column (4.6 mm×50 mm, 1.8 μm) with a 100% mobile phase (A) water-formic acid (100:0.1, v/v/v) to 100% B acetonitrile-formic acid (100:0.1, v/v/v) during 35 minutes.

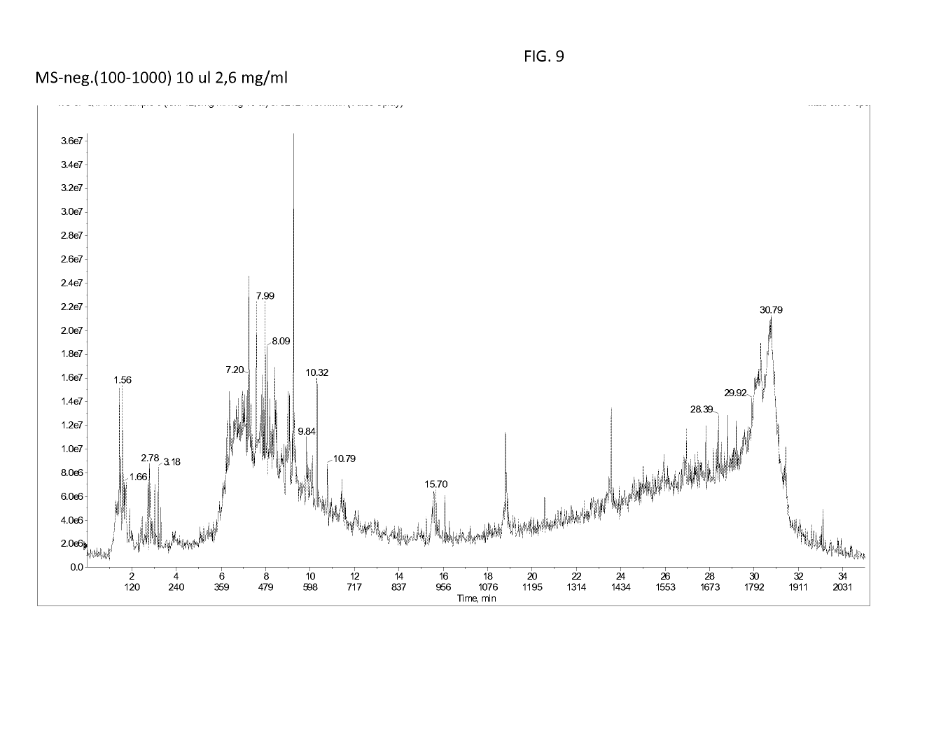

25. The composition of claim 24, wherein said extract exhibits major total ion current chromatogram peaks as observed in FIG. 9.

26. A syrup or solution comprising the composition of claim 1.

27. A powder comprising the composition of claim 1.

28. An oral delivery vehicle comprising the composition of claim 1.

29. A functional food or foodstuff comprising the composition of claim 1.

30. The functional food or foodstuff of claim 29, wherein said functional food or foodstuff is selected from the group consisting of beverages, baked goods, puddings, dairy products, confections, snack foods, frozen confections or novelties, prepared frozen meals, candy, snack products, soups, spreads, sauces, salad dressings, prepared meat products, cheese, and yogurt.

31. A nutritional supplement comprising the composition of claim 1.

32. The nutritional supplement of claim 31, wherein said nutritional supplement is selected from the group consisting of soft gel capsules, hard shell capsules, chewable capsules, health bars, and supplement powders.

33. A method of preventing or treating a disease state initiated or characterized by platelet activation and/or aggregation, improving or maintaining heart health, improving or maintaining cardiovascular health, improving or maintaining circulatory health, or improving or maintaining blood flow in a subject comprising administering to said subject a composition according to claim 1.

34. The method of claim 33, wherein said administering inhibits platelet aggregation.

35. The method of claim 33, wherein said administering results in anti-thrombotic activity.

36. The method of claim 33, wherein said administering results in blood thinning.

37. The method of claim 33, wherein said administering results in reduced blood pressure.

38-39. (canceled)

40. A process for producing a stable and biologically active Actinidia extract comprising producing an Actinidia extract, heating the Actinidia extract under conditions such that the extract retains biological activity during storage, and de-sugaring the Actinidia extract before or after said heating.

41. The process of claim 40, wherein said de-sugaring is performed by a process selected from the group consisting of solid-phase extraction, fermentation, enzyme treatment and nanofiltration.

42. The process of claim 40, wherein said heating comprises heating said fraction to about 70 to about 100 degrees Celsius for greater than about five minutes.

43. The process of claim 40, wherein said Actinidia extract is produced by sedimenting an Actinidia juice or homogenate either before or after heating to provide a sediment fraction and a supernatant fraction, and retaining said supernatant fraction to provide said biologically active Actinidia extract.

44. The process of claim 43, wherein said sedimentation comprises centrifugation at least 3000 g.

45. The process of claim 40, wherein said extract is additionally processed by ultrafiltration either before or after heating.

46. The process of claim 45, wherein said ultrafiltration has a cut-off of between 1000-3000 Daltons.

47. The desugared, stable extract produced by the process of claim 40.

48. A method of reducing blood pressure or treating hypertension in a subject comprising:
administering to a subject in need thereof an effective amount of kiwi fruit or kiwi fruit extract, wherein said effective amount comprises greater than about one whole fruit equivalents of kiwi fruit.

49. The method of claim 48, wherein said effective amount comprises greater than about 3 whole fruit equivalents of kiwi fruit.

50. The method of claim 48, wherein said effective amount comprises from about 2 to about 10 whole fruit equivalents of kiwi fruit.

51. The method of claim 48, wherein said effective amount comprises from about 3 to about 5 whole fruit equivalents of kiwi fruit.

52. The method of claim 48, wherein said kiwi fruit extract is selected from the group consisting of concentrates, powders, syrups, and de-sugarized extracts prepared from kiwi fruit.

53. The method of claim 48, wherein said effective amount causes a reduction in blood pressure in said subject when administered over a time frame selected from the group consisting of 1 week, 2 weeks, 3, weeks, 4 weeks, 5 weeks, 10 weeks, 20 weeks, 30 weeks, 40 weeks, and 50 weeks.

54-59. (canceled)
